(19) United States
(12) Patent Application Publication
(10) Pub. No.: US 2007/0265816 A1
(43) Pub. Date: Nov. 15, 2007

(54) System and method for identifying behavioral signatures

(76) Inventors: Gregory Elmer, Phoenix, MD (US); Neri Kafkafi, Baltimore, MD (US)

Correspondence Address: WHITEFORD, TAYLOR & PRESTON, LLP;ATTN: GREGORY M STONE, SEVEN SAINT PAUL STREET, BALTIMORE, MD, 21202-1626, US

(21) Appl. No.: 11/634,068

(22) Filed: Dec. 5, 2006

Related U.S. Application Data
(60) Provisional application No. 60/775,980, filed on Dec. 5, 2005.

(51) Int. Cl. G06G 7/48; A61B 5/00

(52) U.S. Cl. 703/11; 600/300

(57) ABSTRACT

Psychopharmacological properties of new therapeutic drugs and highly heritable behavior patterns of test subjects are identified based on analysis of monitored exploratory movement to identify behavioral signatures. A test subject in a pen is allowed to explore for a period of time, after injecting it with a candidate drug or control vehicle. The test subject's movement is monitored and its locations stored. The locations are analyzed to separate them into behavioral patterns that are defined based on combinations of behavioral feature. Relative frequencies of performing each behavioral pattern are determined. In each pattern, differences between the relative frequencies in the candidate drug and control groups are tested, and only patterns in which this difference is highly significant are retained. The number of behavioral patterns further is reduced based on the relative frequencies and the correlation of behavioral patterns to one another, with the cells left over corresponding to a set of endpoints that identify a behavioral signature of the effect of the drug.

### CROSS REFERENCE TO RELATED APPLICATION

The current application claims priority under 35 U.S.C. §119 to the U.S. Provisional Patent Application Ser. No. 60/775,980, filed on Dec. 5, 2005, which in herein incorporated by reference in its entirety.

### GOVERNMENT LICENSE RIGHTS

The U.S. Government has a paid-up license in this invention and the right in limited circumstances to require the patent owner to license others on reasonable terms as provided for by the terms of Grant No. DA-022407 awarded by NIH.

### FIELD OF INVENTION

The present invention relates generally to statistically grouping data into patterns. More specifically, the present invention is directed to identifying behavioral signatures based on exploratory behavior that can be used in behavioral genetics or drug discovery applications.

### BACKGROUND OF THE INVENTION

Animal models used in psychiatric drug discovery are often developed strictly for their predictive validity. The main purpose of such a model typically is to predict the neuropharmacological properties of novel compounds with a moderate to high degree of sensitivity and specificity. The behavioral endpoint (measure) may or may not be developed with specific regard to the model's face or construct validity. Ideally, an animal model of this nature is amenable to relatively high through-put and focus on behaviors that have the following properties: i) algorithmically definable and automatically measurable; ii) sufficiently common in natural behavior to supply large samples; iii) sufficiently complex to provide a relatively detailed profile of a drug's psychoactive properties (especially those unique to the drug class); iv) resistant to minor environmental changes; and v) replicable across laboratories (i.e., determined largely through genetics and not environment).

In conventional behavioral drug discovery studies, a drug is administered to a test subject, for example, an animal such as a laboratory mouse. The drug-injected test subject is then subjected to a battery of tests. Tests can be specific to a particular behavior or more generic in nature. For example, a test may be specific to measuring anxiety or learning. However, such specific tests are difficult, expensive, and labor intensive to carry out. Tests that are more general in nature, such a measuring increased activity, are easier and less expensive to carry out. However, general tests are less informative.

Many of the currently employed standard behavioral tests in pre-clinical and basic research automatically record large amounts of information-rich data. The application of bioinformatics paradigms, such as exploratory data analysis and data mining, would appear well suited to be employed with such large amounts of data in order to provide further behavioral information. Unfortunately, in most current behavioral tests these data are rarely explored or mined, and are usually used merely for calculating a small set of hardwired cumulative measures, which may fail to detect subtle behavioral effects in knockouts and transgenics (e.g., Grammer, M., Kuchay, S, Chishti, A. & Baudry, M. (2005) Lack of phenotype for LTP and fear conditioning learning in calpain 1 knock-out mice. Neurobiol Learn Mem 84(3), 222-227, which is herein incorporated by reference in its entirety; Perez, F. A. & Palmiter, R. D. (2005) Parkin-deficient mice are not a robust model of parkinsonism. Proc Natl Acad Sci USA 102(6), 2174-2179, which is herein incorporated by reference in its entirety) or effects of genetic manipulation. Therefore, it would be desirable to provide a behavioral testing paradigm for mining and analyzing large amounts of behavioral data using the large number of measures from the testing to isolate subtle and consistent behavioral effects or effects of genetic manipulation.

Behavioral testing that has been employed for the SOD1G93A (SOD1) rat model of Amyotrophic Lateral Sclerosis (ALS), is an example of the above problem. Transgenic rats and mice expressing any of several mutant human SOD1 alleles show many features of human ALS, including adult-onset muscle weakness as well as severe motor neuron loss (Gurney, M. E., Pu, H., Chiu, A. Y., Dal Canto, M. C., Polchow, C. Y., Alexander, D. D., Caliendo, J., Hentati, A., Kwon, Y. W., Deng, H. X. et al. (1994) Motor neuron degeneration in mice that express a human Cu,Zn superoxide dismutase mutation. Science 264(5166), 1772-1775, which is herein incorporated by reference in its entirety; Bruijn, L. I., Miller, T. M. & Cleveland, D. W. (2004) Unraveling the mechanisms involved in motor neuron degeneration in ALS. Annu Rev Neurosci 27, 723-749, which is herein incorporated by reference in its entirety) usually culminating in death by four months of age. These genetic models are widely used for developing and testing treatments (Howland, D. S., Liu, J., She, Y., Goad, B., Maragakis, N. J., Kim, B., Erickson, J., Kulik, J., DeVito, L., Psaltis, G., DeGennaro, L. J., Cleveland, D. W. & Roth-stein, J. D., (2002). Focal loss of the glutamate transporter EAAT2 in a transgenic rat model of SOD1 mutant-mediated amyotrophic lateral sclerosis (ALS). Proc Natl Acad Sci USA 99, 1604-1609, which is herein incorporated by reference in its entirety; Rothstein, J. D., Patel, S., Regan, M. R., Haenggeli, C., Huang, Y. H., Bergles, D. E., Jin, L, Dykes Hoberg, M., Vidensky, S., Chung, D. S., Toan, S. V., Bruijn, L, I, Su, Z. Z., Gupta, P. & Fisher P B. (2005) Beta-lactam antibiotics offer neuroprotection by increasing glutamate transporter expression. Nature 433, 73-77, which is herein incorporated by reference in its entirety). In SOD1 rats the well-described adult-onset of the disease typically occurs around post-natal day (PND) 110. Discovery of putative earlier motor symptoms that could be measured, in any manner including automatically, and reliably in younger animals may enable investigators to develop and test treatments for delaying or even preventing the disease. Moreover, such symptoms may prove useful for contrasting symptomalogies with non-genetic animal models of ALS (Shaw, C. A. & Wilson, J. M. B. (2006) Environmental toxicity and ALS: novel ilnsights from an animal model of ALS-PDC, in Amyotrophic Lateral Sclerosis. Mitsumoto, H., Przedborski, S. and Gordon, P. H., (Eds), New York: Taylor & Francis, 435-448, which is herein incorporated by reference in its entirety). Unfortunately, such early symptoms have not been found by the current behavioral tests being employed. Matsumoto et al., 2006, (Matsumoto, A., Okada, Y., Nakamichi, M., Nakamura, M., Toyama, Y., Sobue, G., Nagai, M., Aoki, M., Itoyama, Y. & Okano, H. (2006) Disease progression of human SOD1 (G93A) transgenic ALS model rats. J Neurosci Res 83 (1), 119-33, which is herein incorporated by reference in its entirety), has recently phenotyped SOD1 mutant rats using several behavioral tests, including righting reflex, inclined plane (for testing grip strength), home-cage and open-field activity, but failed to detect reliable symptoms before PND 100. Moreover, Matsumoto et al., 2006, failed to detect any abnormality in these animals before PND 90 by subjective observations of their behavior. Therefore, it would be desirable to provide a paradigm capable of screening numerous behavioral patterns in order to isolate reliable differences, such as premorbid (<PND 90) in the current example, in behavioral patterns between diseased subjects and normal subjects. It would be further desirable to utilize these reliable differences in behavioral patterns for contrasting symptomalogies with non-genetic animal models of various diseases.

The example above further illustrates another typical problem with the current behavioral test methodologies being employed, wherein in animal models, the most immediate hypotheses regarding the behavioral effect of the mutation were already exhausted. Using the standard behavioral testing models, the next step may be the testing of a more elaborate hypotheses, in a one-by-one manner using dedicated (and likely costly and time-consuming) setups with an unknown chance of success. Therefore, it would be desirable to provide a behavioral testing paradigm capable of more effectively utilizing the wealth of dynamical motor pattern information, typically collected from simple open-field test of these animals, that to date have mostly been ignored.

Another problem with prior methodologies using animal behavioral models is that the results may be laboratory or experimenter dependent. That is, different results may be obtained simply because of where the testing is performed or who performs the test. Therefore, it would be desirable to provide a behavioral testing paradigm capable of providing more consistent and reliable results regardless of where the testing is performed or who performs the test.

Medications for treatment intervention in drug abuse are currently a significant need for many thousands, if not millions, of people around the world. In addition to the toll drug addiction takes on the individual and those closest, drug addiction costs billions of dollars in direct and indirect health care resources. These costs often being passed on to others through higher premiums. Significant advances in our understanding of the neural mechanisms that underlie drug-taking behavior have been made. Unfortunately, similar advances in developing pharmacotherapeutic interventions have yet to be realized, especially in psychomotor stimulant abuse. Therefore it would be desirable to provide a system and method for discovering the psychopharmacological properties of a durg and to apply it to the study of novel therapeutic agents.

### SUMMARY OF THE INVENTION

Accordingly, the present invention provides a paradigm for behavioral testing capable of effectively utilizing large amounts of data, which may be collected from various behavioral tests (e.g., open-field maze, the photobeam box, the plus maze and the water maze) and performed in various locations and by various people, and identifying statistically significant behavioral patterns. These behavioral patterns (endpoints) may then further be utilized to provide a behavioral signature for classifying drugs, drug discovery, disease diagnosis and/or the testing of therapeutics. In presently preferred exemplary embodiments of the present invention a system and method are provided for discovering the psychopharmacological properties of a drug, for studying new therapeutic drugs, and for identifying highly heritable behavior patterns. Based upon exploration (mining and analysis) of a raw database, exemplary embodiments of the present invention are capable of screening a very large number of potential behavioral endpoints and identifying those that maximize behavioral properties such as those set forth above. For example, in a presently preferred embodiment of the present invention, the constellation of such behavioral endpoints may be used for the development of a psychopharmacological ‘fingerprint’ or behavioral signature for known and novel psychoactive compounds. The ‘fingerprint’ may serve as a valuable tool in classifying known drugs and drug discovery and may lead to broadening of our understanding of the behavioral effects of drugs.

The model is based upon the integration of three concepts. First, exploratory behavior is a complex behavior that is rich in behavioral information. Second, exploratory behavior is amenable to algorithmic structuring. That is, there are highly structured behavioral repertoires amenable to mathematical description. Third, the highly structured repertoires are defined in large part by complex ‘hard-wired’, highly heritable, mechanisms in the brain. As a result, the effects of drugs on this ‘hard-wired’ system which are exemplified by the behavioral patterns (endpoints) of the test subjects are also amenable to algorithmic structuring and identification.

In a presently preferred embodiment of the present invention, the paradigm is capable of identifying behavioral signatures for drug discovery. The raw data (data points) are the path coordinates of a test subject (for example, a laboratory mouse or rat) allowed to explore an “area” (e.g., field, pen or cage) for a period of time while under the effect of a candidate drug, and recorded using a tracking technology, such as video and other tracking technologies as contemplated by those of ordinary skill in the art. In conventional methodologies, these data would be summarized using a small number of commonly-used endpoints. However these endpoints may be far from suitable for best capturing the behavioral effect of the candidate drug.

Embodiments of the present invention, on the other hand, code each path coordinate using a plurality of defined features in order to plot a corresponding endpoint within a cell of a feature space. The range of values in each feature is partitioned into one or more intervals, thus dividing the feature space into a large number (typically tens to hundreds of thousands) of “cells.” Each cell corresponds to a unique combination of features (i.e., a unique behavioral pattern). The effect of the candidate drug on the relative frequency of staying in each cell (i.e., of using this behavioral pattern) is then statistically tested. The very large number of behavioral patterns, identified as the plotted endpoints within the cells of the feature space, highly increases the likelihood that one or more of the patterns may be found to best capture the effect of a specific drug. Thus, the one or more of these pattern frequencies may serve as a set of endpoints that identifies the behavioral signature of the candidate drug.

In a presently preferred embodiment, the present invention provides a method for identifying behavioral signatures, which may be employed for classifying patterns of a known drug(s) and drug discovery. The method includes identifying a set of data points corresponding to a physical location of an exploratory path of a test subject. A plurality of unique behavioral patterns are defined as corresponding to a plurality of features and an interval for each of the features. Each data point is associated with one of the plurality of behavioral patterns, thereby creating an endpoint. The relative frequency for each of the plurality of behavioral patterns is determined and a set of endpoints is identified. From this set of endpoints a behavioral signature may be defined.

In another presently preferred exemplary embodiment of the current invention, a method for identifying behavioral signatures for drug discovery includes monitoring an exploratory path of a test subject injected with this drug, storing locations corresponding to the exploratory path, defining a plurality of features corresponding to behavior, dividing a range of feature into one or more intervals, defining a plurality of behavioral patterns, each behavioral pattern corresponding to a unique combination of intervals of the features, determining a relative frequency for each behavioral pattern, associating each behavioral pattern with an endpoint to form a set of endpoints, and identifying the behavioral pattern with an endpoint to form a set of endpoints, and identifying the behavioral signature as the set of endpoints.

In another particularly preferred embodiment, the present invention is a system for identifying behavioral signatures. The system includes a video camera for monitoring locations explored by a test subject allowed to explore an area for a period of time, a computer communicatively coupled with the video camera and including a storage device for storing the monitored locations. The computer may further be used for determining a relative frequency for a plurality of behavioral patterns, each behavioral pattern corresponding to a unique combination of defined intervals of a plurality of features corresponding to behavior and associated with each of the behavioral patterns an endpoint to form a set of endpoints that act as the behavioral signature.

A particularly preferred embodiment of the present invention provides a method of drug discovery including the step of obtaining an unknown drug and determining the behavioral signature of the unknown drug. Upon determining the behavioral signature of the unknown drug it is compared against the behavioral signature of a known drug. In the final step of the current method the unknown drug is classified based upon a significant correlation between the behavioral signature of the unknown drug and the behavioral signature of the known drug.

Utilizing the behavioral signature identification methodology proposed above, it is another particularly preferred embodiment of the current invention to provide a system for characterizing, including the identification of the effects of the drug and classifying its psychopharmacological profile, novel psychoactive drugs through comparison against known behavioral signatures (psychopharmacological profiles) of known drugs. The system includes a repository of data, including the psychopharmacological profiles of a plurality of drugs. The system further comprises a comprises a computer that is communicatively coupled with the repository and capable of processing, from the input of a novel psychopharmacological profile of a novel drug, the performance of a comparison of the novel drug's profile against the profiles stored in the repository.

### BRIEF DESCRIPTION OF THE DRAWINGS

The numerous advantages of the present invention may be better understood by those skilled in the art by reference to the accompanying figures.

FIG. 1 is an illustration of a block diagram representing a method of employing the current invention in the performance of SEE analysis in accordance with an exemplary embodiment of the present invention.

FIG. 2 is a schematic diagram of a system employed in performance of the current invention in accordance with an exemplary embodiment of the present invention.

FIG. 3 is an illustration of a block diagram representation of a method of isolating candidate behavioral patterns according to an embodiment of the present invention.

FIG. 4 illustrates an exemplary three-dimensional feature space D×V×H that includes vectors of path data points in the form (d, v, h) for 3 mouse genotypes.

FIG. 5 is an illustration of a block diagram representation of a method for reducing the level of cross-pattern correlation using a recursive procedure according to an exemplary embodiment of the present invention.

FIG. 6 is an illustration representing results from 21 different patterns determined in accordance with an exemplary embodiment of the present invention.

FIG. 6A illustrates relative frequency for a first endpoint identified by an embodiment of the present invention in laboratory mice under the effect of several drugs.

FIG. 6B illustrates relative frequency for a second endpoint identified by an embodiment of the present invention in laboratory mice under the effect of several drugs.

FIG. 7 is an illustration of a block diagram representation of a method for diagnosing the presence of a disease in a subject in accordance with an exemplary embodiment of the present invention.

FIG. 8 is an illustration of a block diagram representation of a method for testing the effectiveness of a therapy for a disease in accordance with an exemplary embodiment of the present invention.

FIG. 9 is an illustration representative of path plots from a SOD1 rat (left) and a control rat (right) in the open-field arena. Only progression (movement) segments are shown. Each data point represents 1/30 seconds. The coordinates of these points are the input for the Pattern Array method.

FIG. 10 is an illustration of a 3-Dimensional feature spaces of the same path plots from FIG. 9. Each point in the path plot corresponds to a point in the feature space. The three features chosen here are the distance from the arena wall (d), the acceleration (a) and the curvature of the path (c₄). Grid lines show the division of the feature space into “cells”. Points falling into one of the cells are highlighted (orange). Dividing their number by the total number of points gives the relative frequency of performing this pattern. The highlighted cell here is P{1,*,1,*,*,4,*,*,*}, which is the pattern that best differentiated the SOD1 mutants from the controls.

FIG. 11 is an illustration representing results from SOD1 mutants (closed squares) and Sprague-Dawley controls (open squares) in nine different measures including patterns P{1,*,*,*,*,*,*,*,*}, P{*,*,1,*,*,*,*,*,*}, p{*,*,*,*,*,4,*,*,*} and the discovered pattern P{1,*,1,*,*,4,*,*,*}Animals were divided into two batches, batch A (n=7) and batch B (n=5). Each batch was tested at the ages of 50 days and 80 days old. All results show group means and SEs. * p<0.05; ** p<0.01; # p<0.0000042 (Bonferroni criterion at a level of 0.05 for the mining set). Note that in the P{1,*,1,*,*,4,*,*,*} graph (bottom right), batch A in the 50 days age (diamonds instead of squares) was used as the mining set for discovering the pattern itself.

FIG. 12 is an illustration representing a path plot (left) and speed profile (right) of a Sprague-Dawley rat performing the discovered pattern P{1,*,1,*,*,4,*,*,*}. Each data point represents 1/30 seconds and the six points belonging to the pattern are bolder. The arc in the path plot denotes the arena wall and the circle represents the rat, going from top to bottom of the graph. Notice the turn out of the wall in the path plot and the strong deceleration (negative slope) in the speed profile.

FIG. 13 is an illustration of a block diagram representation of a method of drug discovery in accordance with an exemplary embodiment of the current invention.

FIG. 14 is an illustration of a system for characterizing novel drugs based upon their psychopharmacological profile in accordance with an exemplary embodiment of the current invention.

FIG. 15 is an illustration outlining an approach to the characterization of a drugs effect based upon a three tier systematic approach in accordance with an exemplary embodiment of the current invention.

### DETAILED DESCRIPTION OF THE INVENTION

Embodiment of the present invention screen a large number of complex behavior patterns in the output of a behavioral test and isolate a small set of patterns that maximize some desirable properties, such as discriminating between the experiment groups. Conventional tests for mouse and rat behavioral phenotyping typically export a small set (less than 100) of measures (“endpoints”) that are thought to be relevant for certain aspects of central nervous system (CNS) activity. For example, open-field and “locomotor behavior” tests typically export endpoints such as the distance traveled (“activity”, reflecting some type of “emotionality” and related to dopaminergic drugs) and the center time (thigmotaxis, reflecting relative anxiety). However, the performance of such endpoints (i.e., their ability to differentiate drugs, doses, genotypes, treatments, etc.) is generally very limited, and the results may be highly sensitive to confounding factors such as the laboratory or the experimenter.

Relatively little research has been undertaken to isolate behavioral measures that may perform better than such “traditional” endpoints. However, the standard automated tracking systems used in the open-field tests record the whole path taken by the test subject. The entire path data reflecting the entire path taken by the test subject includes a wealth of complex dynamical patterns of the test subject's movement in the arena, and technically can easily be exported. In conventional systems, however, the entire path is typically not used, except for calculating the above-mentioned small set of traditional measures.

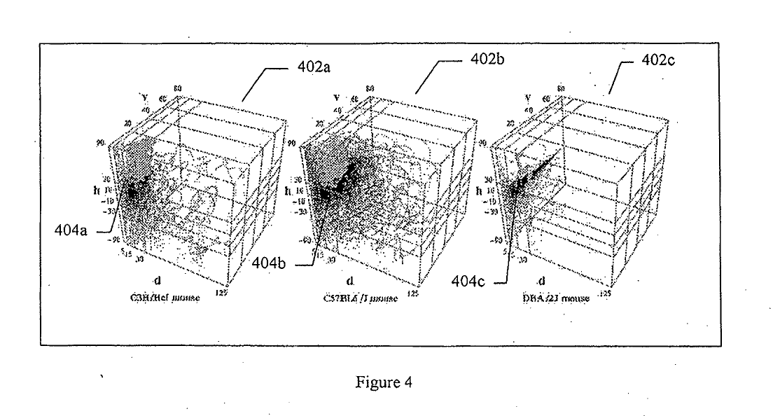

In a preferred embodiment of the present invention a method for identifying behavioral signatures is provided. The method includes tracking and plotting the locations/positions (“data points”) of a test subject's movement within a defined space (“arena”) over a defined period of time. It is to be understood that the test subject may have been administered a known or unknown psychopharmacologically active compound or be operating under an alternative but known set of conditions. A feature space is created as an n-dimensional model (three dimensional model is illustrated in FIG. 4) defined by numerous cells corresponding to n (one or more) particular features and a particular interval within the range of each feature. Each of the cells thereby representing a behavioral pattern (“pattern”) as defined by the features and intervals for those features. Each of the data points is classified into one or more of the various features and feature intervals. This classification allows the data point(s) to be plotted as “endpoints” within a cell of the feature space. Thus, the data points (physical locations/positions of the subject while moving within the arena) are associated with a pattern defined by this cell.

As previously stated, there may be numerous endpoints within the feature space defining numerous patterns. The current invention allows for the statistical analysis of the data points to determine the relative frequency of the cells/patterns. As will be described below, the relative frequency of the patterns allows the current invention to determine those patterns which are statistically significant. These statistically significant patterns may then be used to form the “behavioral signature” exhibited by the subject under the conditions specified.

Thus, in contrast to the behavioral testing methodology stated above, preferred embodiments of the present invention make use of entire-path data by defining a large number (typically around 100,000, but more or less is contemplated) of potential behavior patterns (“endpoints”), and isolating certain endpoints based upon the novel paradigm of the current invention. Those endpoints may maximize the properties set forth above and once isolated may be easily measured in any laboratory using standard tracking systems and simple stand-alone programs. Such isolated endpoints have varied potential applications, depending on the experimental groups for the dataset that was used to generate them.

For example, in one embodiment of the present invention, the isolated endpoints may be used to identify highly heritable behavior patterns. When the experimental groups in the dataset includes different genotypes (inbred strains), the isolated patterns reveal what components of behavior are the most controlled by the genotype. An exemplary embodiment of the present invention described below uses a dataset of 10 inbred strains across three test facilities to demonstrate such an application.

In another embodiment of the present invention, the isolated endpoints may be used to develop animal models for drug classification and discovery. When the experimental groups in the dataset includes animals treated with psychoactive compounds, the isolated patterns are good candidates for use as animal models of psychiatric disorders and may allow for the pattern classification of the compound, and may be used for predicting the effect of new or additional drugs. An embodiment of the present invention using a small dataset including anxiogenic and anxiolytic drugs is described below to demonstrate such an application.

Embodiments of the present invention screen a large number of endpoints, possibly hundreds of thousands of endpoints or more. One problem with screening such large numbers of data points is that it raises a multiple comparisons problem. While brute force comparison methods could be used according to embodiments of the present invention, additional techniques for reducing the data sets are available. For example, in one embodiment of the present invention use of a multiple comparison criterion, such as the Bonferroni criterion or the false discovery rate may be employed. A description of the false discovery rate is described in Y. Benjamini et al., Controlling the False Discovery Rate in Behavior Genetics Research, BEHAV. BRAIN RES., at 279-84 (2001), which is hereby incorporated herein by reference in its entirety.

In another embodiment of the present invention, a cross-validation approach may be used whenever possible. In cross-validation, the best endpoints are isolated using one set of data (the “training set”) and then their performance is evaluated in another set of data (the “test set”).

As previously stated, preferred embodiments of the present invention may be applied to various behavioral tests that can export large amounts of data. One such behavioral test is the open-field test using “Software for the Exploration of Exploration” (SEE open-field test). The SEE open-field test is described in D. Drai. & I. Goilani I, SEE: A Tool for the Visualization and Analysis of Rodent Exploratory Behavior, NEUROSCI. BIOBEHAV. REV., at 409-426 (2001), which is hereby incorporated by reference in its entirety. The SEE open-field test offers several advantages for animal behavioral studies, including the following:
1. “Open-field” and “locomotor behavior” are well-established behavioral tests in both psychopharmacology and behavior genetics, with both rats and mice, and are considered relevant for several main drug classes.
2. The SEE open-field test can be performed in a relatively high-throughput manner, using automatic tracking that is already available in many laboratories.
3. The SEE open-field tests produces a large amount of data, for example, 54,000 data points (x,y coordinates of the path in the arena) per single 30 minute session when tracked in a rate of 30 measurements per second.
4. The path data generated by the SEE open-field test is of high resolution and quality, especially after the tracking noise is filtered using robust smoothing algorithms that were specially developed for the SEE open-field test. An example of such filtering is described in Hen, et al., The Dynamics of Spatial Behavior: How Can Robust Smoothing Techniques Help?, JOURNAL OF NEUROSCIENCE METHODS, at 161-72 (2005), which is hereby incorporated by reference in its entirety.
5. The data generated by the SEE open-field test has high information content: it faithfully captures complex nuances of movement in the arena, including dynamic properties such as the momentary speed, momentary acceleration and even higher derivates.
6. In contrast with the common view of open-field behavior as mainly stochastic in nature, a series o ethological studies in both rats and mice has shown that this is a complex exploratory behavior including a wealth of typical patterns. The SEE open-field test is suitable for studying this exploratory behavior.
7. Several exploratory patterns generated by the SEE open-field test have been shown to be reproducible across laboratories. Further, the issue of genotype×environment interaction has been discussed in the literature. For example, such a discussion is found in N. Kafkafi et al., Genotype-environment Interactions in Mouse Behavior: A Way out of the Problem, PROC. NATL ACAD. SCI., USA at 4619-24 (2005), which is hereby incorporated by reference in its entirety.
8. The SEE open-field test software was specifically developed for studying behavioral patterns. It is an advanced analysis tool embedded in the programming environment of Mathematica™, which offers a large selection of advanced mathematical functions and algorithms.
9. A large database of SEE open-field test path data from more than 1000 test subjects across several inbred lines, knockouts, drugs and laboratories exists. The SEE open-field test program has been extended to enable SEE to retrieve any desired part of the path from any cross-section of this database, as for example described in N. Kafkafi et al., SEE Locomotor Behavior Test Discriminates C57BL/6J and DBA/2J Mouse Inbred Strains Across Laboratories and Protocol Conditions, BEHAV. NEUROSCI. At 464-77 (2003), which is hereby incorporated by reference in its entirety.

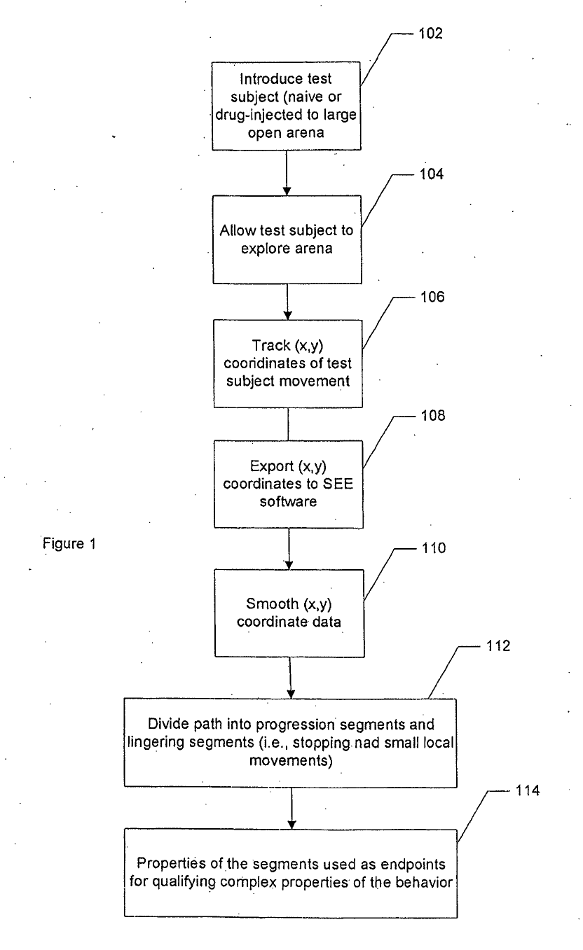

FIG. 1 summarizes the principles of SEE analysis. In step 102, the test subject, such as an animal (naïve or drug-injected), is introduced to a large open-field arena or pen. In step 104, the test subject is allowed t explore the open-field arena for an amount of time (e.g., 10-90 minutes). In step 106, the x,y coordinates of the path traversed by the test subject are tracked, and exported to the SEE software in step 108. Tracking can be performed automatically by digitizing its location using a tracking system. In step 110, the data are smoothed to filter tracking noise. In step 112, the path traversed by the test subject is divided into progression segments and lingering episodes (i.e., stopping and small local movements). Progression and lingering episodes can be divided using a categorization demonstrated to be inherent to the behavior as described in D. Drai, et al., Rats and Mice Share Common Ethologically Relevant Parameters of Exploratory Behavior, BEHAV. BRAIN RES. At 133-40 (2002), which is hereby incorporated by reference in its entirety. Embodiments of the present invention use properties of these segments, such as length, duration, speed and acceleration are used to derive patterns called “endpoints” for quantifying complex properties of the behavior. The SEE software is well known and details of its operation are publicly available.

The usefulness of SEE analysis is demonstrated by the results of two recent studies performed by the inventors of the present invention. First, N. Kafkafi, et al., Genotype-Environment Interactions in Mouse Behavior: A Way Out of the Problem, PROC. NAT'L. ACAD. SCI. U.S.A., at 4619-24 (2005), which is hereby incorporated by reference in its entirety, describes a study that obtained and analyzed the exploratory behavior in 8 different inbred strains in three different laboratories in two different countries to determine, in part, the degree to which SEE is capable of identifying robust behavioral traits that are relatively stable across different environments. The study identified 9 out of 17 behavioral endpoints that had heritability rates higher than 50% (most behavioral measures are below 50%) despite the disparate lab conditions. Moreover, the method was able to identify behavioral endpoints that consistently differed between genotypes in a reliable and replicable manner.

In a second study, described in N. Kafkafi & g. Elmer, Texture of Locomotor Path: A Replicable Characterization of a Complex Behavioral Phenotype, GENES BRAIN BEHAV., at 431-43 (2005), which is herby incorporated by reference in its entirety, an ‘in silico’ strategy was used to search an existing database of mouse exploratory behavior for novel behavioral measures that could discriminate between genotypes and pharmacological treatments. This database included the data described in the previous experiment and several pharmacological treatments. The new behavioral measure, “path texture”, was characterized using the curvature of the path during progression across several distance scales, starting from scales smaller than the animal's body length and up to the scale of the arena size. This ‘in silico’ discovered a novel behavioral endpoint, found to discriminate genotypes with high replicability across laboratories (72% heritability rate at intermediate scale).

In the pharmacological studies, for example, this endpoint was able to qualitatively discriminate the effects of amphetamine between two different genotypes (DBA/2J vs. C57BL/6J); amphetamine decreased the path curvature of C57BL/6 mice while having no effect on BDA/2J mice (despite a 3-fold increase in distance traveled in both strains). In the database having only C57BL/6J mice, diazepam dose-dependently decreased the curvature while two anxiogenic drugs, FG 7142 and pentylenetetrazole, increased it.

In the above studies, novel behavior endpoints were defined from watching the actual behavior and/or several types of graphic visualization in SEE. Once a behavioral pattern was algorithmically defined, it was tested on the SEE database in order to evaluate advantageous properties such as heritability and replicability.

Embodiments of the present invention use a more robust technique rooted in ethological building blocks for determining endpoints. Rather than require a developer of a new behavioral endpoint to explicitly define it as a behavioral pattern, a developer employing an embodiment of the present invention defines a short list of simple “features” that are considered relevant for the behavior. These features can be different for each behavioral test. In the SEE open-field test, for example, features such as the momentary curvature of the path, the momentary direction of movement, the momentary distance from the wall and the momentary speed are used. The time-series of the path coordinates is thus transformed into a time series of feature vectors.

Each feature is partitioned into a small number of intervals. Thus, the space of the features is partitioned in many (typically, more than 100,000) “cells.” Each of these cells corresponds to a movement pattern—for example, located a certain distance from the wall while turning in a certain direction at a certain speed, and the like, and, therefore corresponds to a behavioral pattern. These patterns are subsequently screened for those that maximize advantageous properties such as high heritability, high pharmacological specificity, low cross-laboratory variance (when applicable) and low cross-trait correlation. A stringent multiple comparisons criterion is applied during this process to avoid false positives. For increased reliability, the identified patterns can be cross-validated using data that was not used to derive them.

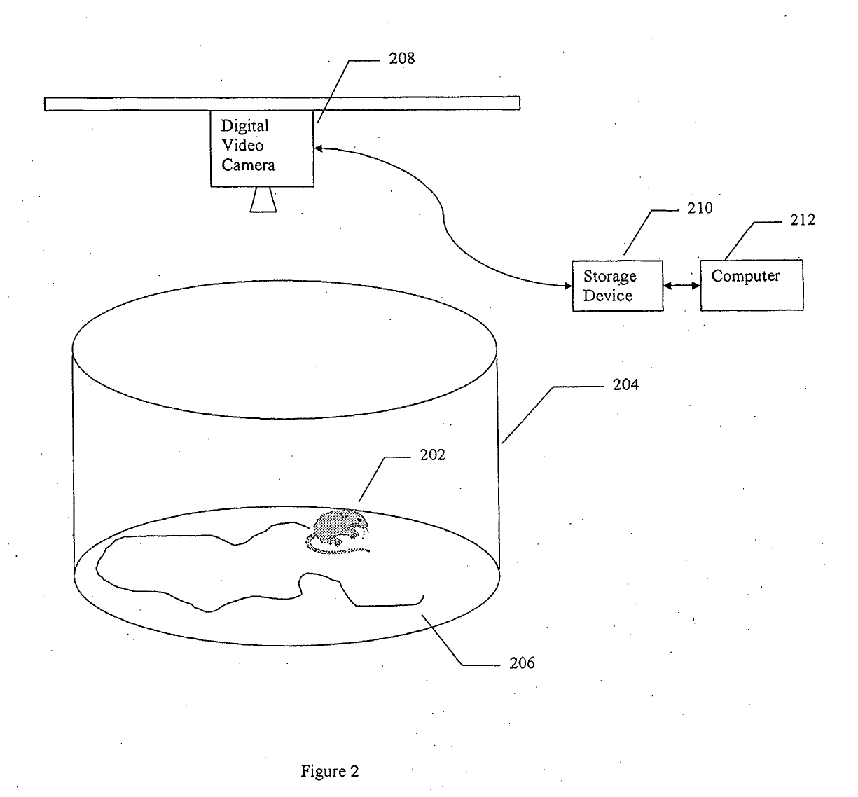

FIG. 2 is a schematic diagram of an exemplary test set up according to an embodiment of the present invention for monitoring a test subject's movement in a pen. A test subject 202 moves in a path 206 in a pen or arena 204. The test subject may be any test subject whose movements may be monitored. For example, the test subject may be an animal such as a mouse or rat. In an embodiment of the present invention, pen 204 is cylindrical with a 2.5 meter circumference. A video camera 208 captures the test subject's movements and stores them on a storage device 210 associated with a computer 212. It is contemplated that the capture and storage of the subject's movements may occur utilizing various alternative technologies as may be contemplated by those of ordinary skill in the art. Video camera is preferably a digital camera with at least a 1 cm resolution for observing the test subject's movements about pen 204. Storage device 210 may be any storage device including any internal or external disk drive whether a floppy disk or a hard disk, and can be removable or fixed.

It is contemplated that the method of embodiments of the present invention may be computationally expensive. Data in embodiments of the present invention may be retrieved from a large (several Gbytes in size) database of behavioral data from hundreds of animals. In a preferred embodiment, computer 212 may be workstation with a fast processor (e.g., a 64-bit and possibly dual-core processor) and two hard drives in a RAID configuration for fast retrieval. An exemplary preferred computer is a Dell Precision 380 MT64 workstation with a Pentium EE processor, 3 GB memory and RAID configuration configured to execute Mathematica™ software. The Mathematica™ software is available from Wolfram Research of Champaign, Ill. Alternative data retrieval technologies may be employed without departing from the scope and spirit of the present invention.

Two exemplary embodiments of the present invention are described below. In the first exemplary embodiment, the present invention is used in a behavior genetics application. In the second exemplary embodiment, the present invention is used in a pharmacological application.

Turning to the first exemplary embodiment of the present invention, when the experimental groups of the database include different genotypes, embodiments of the present invention may be used to identify specific behavioral traits that are highly heritable. When the database further includes results from different laboratories, embodiments of the present invention may also identify traits that are more replicable and robust to small environmental changes.

Behavioral data for the exemplary embodiments was gathered using the SEE open-field test described above. A mouse was introduced to a 2.5 meter circular pen and allowed to freely explore the pen for 30 minutes while the mouse's location was video-tracked and digitized at a rate of 25-30 Hz with a spatial resolution of approximately 1 cm. The tracked coordinates are stored on a storage device and imported into the SEE software. Using SEE software procedures the path is further smoothed and segmented into stops and progression segments. The data file of each animal thus included 45,000-54,000 data pints of its coordinates in the arena. Because small, local movements during stopping cannot be properly captured using available tracking technology, the analysis was limited to progression segments. Depending on the activity of the test subject, progression segments generally contain 3,000-30,000 data points (100-1000 s) per animal. Lingering segments may also be used with tracking equipment having sufficiently fine resolution to see the small, local movements associated with lingering segments.

The dataset used for the exemplary embodiments of the present invention include 10 mouse inbred strains: BALB/cByJ, C3H/HeJ, C57BL/6J, DBA/2J, FVB/NJ, SJL/J, 129S1/SvImJ, CAST/EiJ and DZECHII/EiJ, all of them included in the priority strains of the Jackson Laboratory Mouse Phenome Database. The CAST/EiJ and CZECHII/EiJ are wild-derived inbred strains that were included to increase the genetic diversity. All strains were tested across three test facilities: National Institute of Drug Abuse—IRP in Baltimore (NIDA), Maryland Psychiatric Research Center, University of Maryland (MPRC), and the Dept. of Zoology in Tel Aviv University (TAU). This experiment was used in N. Kafkaifi et al., Genotype-Environment Interactions in Mouse Behavior: A Way Out of the Problem, PROC. NATL. ACAD. SCI. U.S.A. at 4619-24 (2005), which is hereby incorporated by reference in its entirety, to evaluate the replicability of genotype differences using conventional behavioral measures. The number of test subjects in each group of strain-in-batch-in-lab was usually 6. To estimate replicability across laboratories the testing was done using Mixed Model ANOVA with the laboratory as a random factor as described, for example, N. Kafkaifi et al., Genotype-Environment Interactions in Mouse Behavior: A Way Out of the Problem, PROC. NATL. ACAD. SCI. U.S.A. at 4619-24 (2005).

Embodiments of the present invention use a 5-step method that employs ethologically-based building blocks to “mine” the data for novel behavioral patterns (endpoints) that maximize desired properties including, for example, high heritability and replicability across laboratories. Cross-validation is preferably employed to increase confidence in discovered endpoints. In this case, the first batch of test subjects (across all three labs) was used as the “Training Set.” Novel patterns (“endpoints”) are identified from analysis of the data generated during testing of the training set. A second batch of test subjects (again across all labs) was used as the “Test Set.” The identified endpoints are tested using data generated during testing of the test set.

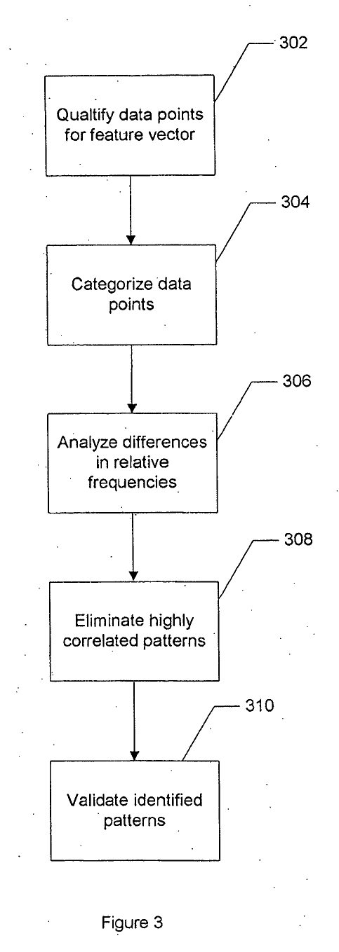

FIG. 3 is a flow chart for a method of isolating candidate behavioral patterns according to an embodiment of the present invention. In step 302, the data points are quantified for a feature vector. In the embodiment of the present invention being described, each data point observed during the animal progression is quantified using seven “features” (variables) (t, d, v, a, j, h, c). The seven features are relevant to open-field behavior and genotype differences. Other features, or different subsets of the described features may be used in other applications of the present invention. The 7 features used in the current example are described in Table 1.

TABLE 1
Basic behavioral variables

| Symbol | Feature | Unit | # of intervals | Interval boundaries |
|---|---|---|---|---|
| t | Time from beginning of | min | 3 | 0, 10, 20, 30 |
| | session | | | |
| d | Momentary distance from | cm | 4 | 0, 5, 15, 30, 125 |
| | arena wall | | | |
| v | Momentary speed of | cm/s | 4 | 0, 20, 40, 60, >60 |
| | movement | | | |
| a | Momentary acceleration of | cm/s² | 5 | <−30, −30, −10, 10, 30, >30 |
| | movement | | | |
| j | Momentary jerk of movement | cm/s³ | 5 | <−300, −300, −100, 100, |
| | | | | 300, >300 |
| h | Momentary movement | degrees | 5 | −90, −30, −10, 10, 30, 90 |
| | direction relative to wall | | | |
| c | Momentary path curvature | degrees/cm | 5 | <−5, −5, −1, 1, 5, >5 |

In the present exemplary embodiments of the present invention, the range of each feature is divided into several disjoined intervals, using the boundaries detailed in the rightmost column of Table 1. The 7-dimensional feature space is thus divided into 30,000 “cells”. The number of cells may be chosen based on the known resolution in each feature and the total number of cells not being too high (i.e., there are sufficient data points per cell for statistical testing). The intervals are not necessarily equal-size, but rather chosen to contain approximately equal frequencies of data points. The size of the intervals may be determined for example using histograms from several test subjects. For example, mice from all strains move near the wall much more than they move in the center of the arena. Consequently, in an embodiment of the present invention, the distance from the wall, d, was divided into intervals having boundaries of 0, 5, 15, 30 and 125 cm.

In addition, the same process is repeated in all sub-spaces of the 7-dimensional feature space, from one-dimensional spaces, using one-dimensional vectors such as (d), (v) and (c) through two-dimensional, using vectors such as (d, v), (d, a) and (h, c), to six-dimensional, using vectors such as (t, d, v, a, j, h), (t, d, v, a, j, c) and (d, v, a, j, h, c). Thus combinations in which one or more of the seven features are not present are also considered.

For example, FIG. 4 illustrates a three-dimensional space D×V×H that includes vectors of the form (d, v, h) for 3 mouse genotypes while the features t, a, j, and c were disregarded. In FIG. 4, data points populating the sub-space D×V×H are presented as vectors of the form (d, v, h) in three mice from genotypes C3H/HeJ (402a), C57BL6/J (402b) and DBA/2J (402c). Grid lines show the division of the space to cells. Data points falling into one cell are indicated by 404a, 404b and 404c. The number of these points divided by the total number of points, for each mouse, is the relative frequency of this cell for that mouse.

Including all the additional sub-spaces, the total number of cells increased to 129,599. Each cell defines a different pattern of movement that can potentially be named. For example, a cell corresponding to a pattern characterized by walking in slow (low v) uniform speed (a and j both near zero) close to the wall (small d) and parallel to it (h near zero) might be named “wall strolling.” Another cell corresponding to a cell characterized by a high d, high v, near-zero a and very negative j might be named “center speed peaks.” Another cell corresponding to a cell characterized by a high d, high a and very negative h might be named “speeding peals.” Another cell corresponding to a cell characterized by a high d, high a and very negative h might be named “speeding back to wall.”

In step 304, the data points are characterized. For each test subject, the data points of the path belonging to that test subject's progression mode are classified into the cells. That is for each test subject, each time a cell (pattern) is observed in that test subject's progression mode path, the cell (pattern) is given a one up count. When all of the data points corresponding to all of the test subjects' progressions have been categorized in this manner, an array, matrix, or other storage structure will exist wherein each element of the array, matrix, or storage structure corresponds to a cell, and the value of the storage structure element corresponding to a particular cell corresponds to the number of times that cell (pattern) was observed. The number of times a cell pattern is observed is the cell frequency. For each test subject, the cell frequency is divided by the number of overall data points for the test subject spent in the progression mode. The result of the division is a relative frequency for the cell. The relative frequency of the cell corresponds to the fraction out of total progression time spent in the cell. In one embodiment of the present invention, this fraction is represented using the logit transformation.

In step 306, the difference in relative frequencies are analyzed. For each cell, the relative frequencies from all test subjects are tested for strain differences using one-way ANOVA, pooled over laboratories. To correct for multiple comparisons only cells with p-values more significant than the Bonferroni criterion for a level of 0.05/129599=3.86×10⁻⁷ are considered. In addition, only cells in which the mean number of data points per test subject (pooled over labs and strains) was at least 30 (i.e., the test subjects spent on average at least one cumulative second performing this pattern) are considered.

In step 308, highly correlated patterns are eliminated. Within the remaining cells there may still exist a high level of cross-cell correlation. That is, one cell may not add any information to another cell, because all test subjects that had high relative frequency in the first cell also had high relative frequency in the second cell, and all test subjects that had low relative frequency in the first cell also had low relative frequency in the second cell. This is especially true because many of these cells are partially overlapping (as described below).

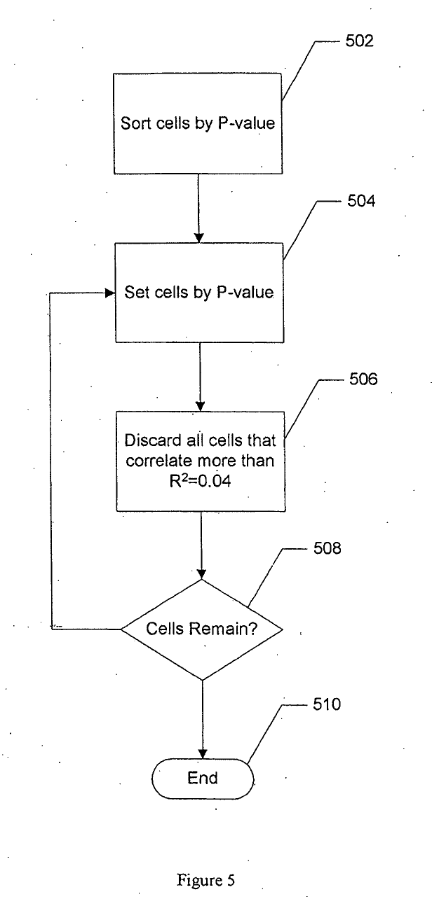

FIG. 5 is a flow chart for a method for reducing the level of cross-trait correlation using a recursive procedure according to an embodiment of the present invention. In step 502, the cells are sorted by p-value. In step 504, the cell having the most significant p-value is stored. In step 506 all cells that correlate more than R²=0.4 with the cell stored in step 504 (as computed over all test subjects in the training set, pooled over strain and lab) are discarded. In step 508 it is determined if there are remaining cells. If there are, the method continues in step 504. If there are no remaining cells, the method ends in step 510. The end result is generally a much shorter list of the most significant cells in which the cross-cell correlation is 0.4 at most.

In step 310, the identified patterns are validated. All the previous steps are performed in the training set. Finally, the remaining list of cells is tested in the test set using mixed ANOVA of the (logit-transformed) relative frequencies in those cells. Mixed ANOVA also tests the replicability of the results across laboratories, since the genotype difference is tested over the Genotype X Laboratory interaction as well as the within group variance. The significance is corrected for multiple comparisons using FDR as described, for example, in Benjamini et al, Controlling the False Discovery Rate in Behavior Genetics Research, BEHAV. BRAIN RES. At 279-84 (2001), which is hereby incorporated by reference in its entirety.

After applying the method of FIG. 3 in the first exemplary embodiment of the present invention, the 129,599 cells were reduced in number to 7,181 after discarding insignificant patterns in step 306. Sixty-nine of the 7,181 cells remained after discarding cross-correlating patterns in step 308.

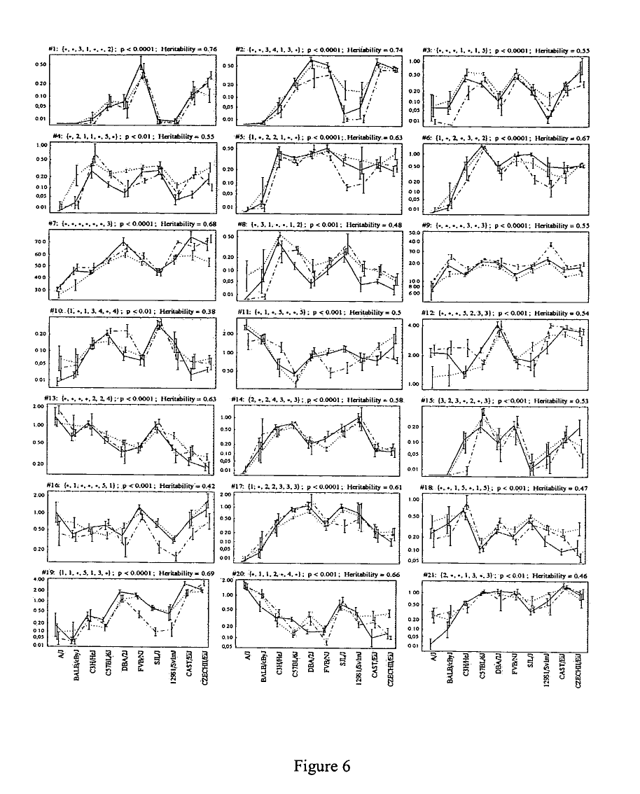

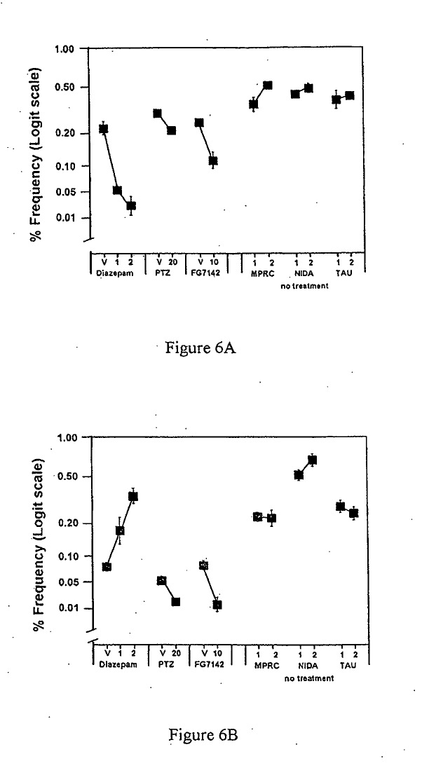

The 69 patterns are applied to the test set. FIG. 6 shows test set results for 21 of the patterns that were the most significant in the training set. Due to space considerations, only 21 of the 69 patterns are illustrated in FIG. 6. As the graphs in FIG. 6 show, the heritability in many patterns is higher than 50%. The replicability across laboratories is usually high (p-values indicating that genotype differences are mostly highly significant in Mixed Model ANOVA). Three of the 10 strains, the A/J and the two wild-derived strains CAST/EiJ and CZECHII/EiJ, were not used in the training set. However, their variability both within and across labs is similar to that of the 7 strains that did participate in the training set. This suggests that these traits have general validity in mouse behavior genetics, not merely in the particular strains in which they were identified. Each of the patterns has a slightly different configuration of strain differences.

Some of the best patterns are narrowly specified, such as #17. Pattern #17 is defined by 6 out of the 7 features and thus corresponds to a very specific pattern of behavior. Other examples of the best patterns are very broadly specified and should be thought of as general properties or qualities of movement rather than specific behaviors. For example, pattern #7 specifies only a single feature: that the curvature of the path will be near zero (i.e., a very smooth path). As the graph shows, most mice display this property during 30-70% of their progression time. In contrast, some of the most significant and heritable patterns, e.g., #1, #2 and #5, account for only 0.5% of the progression time at most and mice from some strains to dot perform them at all.

Because these patterns are defined in many possible resolutions, some of them are partially overlapping, or are subsets that are included in more general patterns. For example, #9 is a sub-set of the above-mentioned #7. In addition to the near-zero curvature it also specifies a near-zero jerk. That is, the progression is also dynamically smooth (“jerk” is the derivative of acceleration, thus near-zero jerk implies no abrupt changes of acceleration). Yet the strain configuration of #9 is different that that of #7. Patterns #14, #17 and #21 are different sub-sets of #9, and they show yet different strain configurations. Therefore, the structure of heritable behavioral differences discovered by the method illustrated in FIG. 4 is actually hierarchical. That is, the feature spaces are either one-dimensional, two-dimensional, three-dimensional (as demonstrated in FIG. 4) or four-dimensional, depending on whether one, two, three or four features are used for specifying a specific pattern.

The estimation of broad-sense heritability was higher than 50% in more than 30 different patterns. As is well known, heritability greater than 50% is considered high heritability in behavior genetics studies. Despite the large number of measures all coming from the same test only three pairs of patterns correlated with R. 0.87 and none with R, −0.8. This improvement in reducing the cross-trait correlation results from using embodiments of the present invention despite that many of the discovered patterns have considerably higher heritability than most traits in conventional MPD projects.

A second exemplary application for the present invention applies the 5-step method of FIG. 3 to identify specific features that describe a drug's behavioral effects. In particular, the second exemplary application of the present invention is used to characterize anxiolytic or anxiogenic properties of a drug. As described above, the method is used to screen more than 100,000 behavioral patterns for those that best differentiate the experimental groups. In the present example, the same behavioral patterns are considered. The only principal difference is that the experimental groups include mice injected with anxiolytic and anxiogenic drugs, rather than mice of different inbred strains. An important corollary application is that the behavioral patterns identified as ‘anxiogenic’ could be used to determine if a non-treated subject is ‘anxious’.

As in the first exemplary application described above, the test subjects in the second exemplary application are mice. The dataset used in this example includes mice (C57BL/6J males 60-80 days old) tested with anxiolytic and anxiogenic drugs. The mice were shipped from Jackson Laboratories and housed in the animal colony in MPRC for at least two weeks before they were tested. Further, the mice were kept in standard conditions of 12:12 light cycle, 22° C. room temperature, water and food ad libitium, and housed 4 per cage. The mice were injected with either diazepam (1 or 2 mg/kg, i.p.), FG-7142 (10 mg/kg, i.p.) or pentylenetetrazole (PTZ, 20 mg/kg,i.p.) and immediately placed in the 2.50 m circular arena. Doses were assigned so that no two mice from the same cage received the same drug and dose. The mice were tested between the hours of 09:00 and 15:00 in 60 minute sessions. There were 4-7 animals in each drug treatment group, for a total of 34. Diazepam was dissolved in saline. FG-7142 (b-Carboline-3-carboxylic acid N-methylamide) was put in solution using 17.5 mM (2-Hydroxypropyl)-b-cyclodextrin (Sigma, St. Louis) and 5% Tween. PTZ was dissolved in saline.

Step 310 was not performed for the second exemplary embodiment of the present invention. Consequently, only training set results are provided below. However, these results were checked against a database of discovered patterns in an independent set of C57BL/6J non-injected animals from the behavior genetics example, which includes two different batches from each of three labs. While this independent set cannot be used to test the drug effect in the discovered endpoints, it offers useful corroboration for some general properties of these patterns and to determine if a set of mice from one of the three labs were more or less anxious (as defined by the discovered behavioral patterns).

Out of the 129,599 possible “cells” or behavior patterns, only 387 patterns remained after analyzing the difference (ANOVA) in step 306, as compared to 7,181 in the inbred strains set. This is to be expected since the group size in the inbred strains set was 18 mice (when pooled over labs) while the group size in the pharmacological set was only 4-7 mice. Thus, the p-values of the ANOVA test have a smaller chance to pass the Bonferroni criterion (p=3.86ˆ10⁷). Out of these 387, only two patterns remained after eliminating highly correlated patterns in step 308, as compared to 69 in the inbred strain data set. The relative frequencies of the occurrence of these two patterns are shown in FIGS. 6A and 6B.

The first pattern detected was (3,*,*,4,1,*,3). FIG. 6A shows the relative frequency of the first pattern. The first pattern discriminates across drug use, but would not between drugs. Consequently, the first pattern likely reflects general drug effect. For example, the first pattern detected the effect of both diazepam and the two anxiogenic drugs. However, the differences from vehicle (control injection) for both the anxiogenic and anxiolytic drugs were in the same direction (decrease) all three drugs significantly reduced the relative frequency of performing this pattern.

The second of the two final patterns was (3,1,4,*,3,5,*). The relative frequency of observations of the second pattern is shown in FIG. 6B. The second pattern discriminates between drugs (anxiolytic vs. anxiogenic) and across drug dose. Therefore, this pattern likely reflects a particular drug effect that is capable of discriminating anxiolytics from anxiogenics. In addition, the endpoint may in turn be used to assess a relative state of anxiety. Injected mice are more anxious than non-injected mice (vehicle treated mice are in the same direction as the anxiogenic drug) and NIDA mice may be less anxious than experimental mice (NIDA mice are in the same direction as the anxiolytic drug). For example, the second pattern detected a dose-dependent effect of diazepam that was opposite in direction to the effect of the two anxiogenic drugs. Given these qualities, the behavior pattern identified by the second application of the present invention has the potential for use as an animal model for discovering anxiolytic drugs.

A set of one or more of patterns that are found, using the above embodiment for drug discovery, to highly differentiate the behavior of different drugs, may be used for generating a “behavioral signature” for each drug. The signature of a drug may be defined as the frequencies in which each of these patterns was performed by the test subjects under the effect of this drug. This signature may also be specified for each dose that was tested for the drug. For example, the “signature” of the drug Diazepam in a dose of 1.0 mg/kg may be defined by two frequencies, those shown for Diazepam 1 mg/kg (second from the left) in FIG. 6A and FIG. 6B. Analyzing data from several drugs in this way provides a “library” or “repository” of signatures for each drug and dose. For example, FIGS. 6A and 6B may be used to specify two-patterns signatures of the drugs Diazepam, PTZ and FG7142 in several doses. The behavior generated by a novel drug with unknown effects may then be characterized by comparing it against this library. For example, if a novel drug is found to generate a signature similar to that of Diazepam, then this novel drug may be implicated for use as a treatment against anxiety.

The non-injected test subjects of the independent set performed the pattern more than the vehicle-injected animals and in the same direction as the effects of diazepam. This result is to be expected given the likely anxiety-provoking effects of the injection procedure itself. In addition, while this pattern was highly replicable within a lab across batches of test subjects run at least one month apart, this pattern was performed at a greater frequency in the NIDA laboratory. This suggests that some factor in the housing or testing in NIDDA made the animals there less anxious than in the other two labs.

This “anxiety dependent” pattern in FIG. 6B is defined by 5 out of the 7 features as follows: during 20-30 min from the beginning of the session, near the arena wall (9-5 cm), low speed, near-zero jerk, and turning away from the wall. That is, the second pattern was (3,1,4,*,3,5,*). The time is again consistent with the kinetics of the drugs. The low speed, the near-zero jerk (i.e., no change in acceleration, the speed changing very smoothly) and the turning away from the wall even when the wall is very near may indicate more confidence in the arena context. Again, the relative frequency of performing this pattern was lower in the test subjects injected with the anxiogenic drugs.

It is contemplated that there may be a particular behavioral pattern associated with the anxiogenic drugs and that this particular pattern may be indicative of an ‘anxious state’. The utility of the application of the current invention to characterize this particular behavior may be further supported through the performance of various standard tests. For example, the subject may be presented stimuli previously paired with noxious stimuli (i.e. shock, fox odor) to the subject in the exploratory arena. In this way it may be determined if the pattern isolated using the anxiogenic drugs is truly reflective of an anxious state as determined by more traditional behavioral methods. Along this same line, the subject may be presented stimuli previously paired with reward in order to characterize behavioral patterns characteristic of reward states.

In general the current invention is providing a step-by-step methodology for determining behavioral signatures. In a preferred embodiment, the methodology may be summarized as a five (5) step process.

Step 1, Quantify data points for feature vector: Each data point during the animal progression may be quantified using n number of features. In the preferred embodiments, 7 “features” (variables) (t, d, v, a, j, h, c) are relevant to open-field behavior and genotype differences. These 7 features are described in Table 1.

Step 2, Categorize data points: In each animal, data points of the path belonging to the progression mode are classified into the cells (FIG. 4). In each animal the cell frequency is divided by the number of overall data points this animal spent in the progression mode, to get the relative frequency, or the fraction out of total progression time spent in this cell. This fraction is represented using the logit transformation.

Step 3, Analyze differences in relative frequencies: For each cell, the relative frequencies from all animals are tested for strain differences using one-way ANOVA, pooled over labs. To correct for multiple comparisons, cells with p-values more significant than the Bonferroni criterion for a level of 0.05, (0.05/129599=3.86×10⁻⁷) are considered.

Step 4, Eliminate highly correlated patterns: Within the remaining cells there is still a high level of cross-cell correlation. That is, one cell may not add any information to another cell. This is especially true because many of these cells are partially overlapping. In order to reduce this level of cross-trait correlation we use a simple recursive procedure as follows: (i) Sort the cells by their p-value; (ii) Keep the cell with the most significant p-value, and discard any cell that correlates more than R²=0.4 with it (as computed over all animals in the Mining set, pooled over strain and lab); (iii) repeat steps ii and iii in the remaining cells, again keeping the most significant remaining cell and discarding any cell correlating with it. This process is repeated until no cells remain, yielding a much shorter list of cells (all with cross-cell correlation ≦0.4).

Step 5, Validation of identified patterns: All the previous steps are performed in the Mining Set. Finally, the remaining list of cells is tested in the Test Set, using mixed ANOVA of the (logit-transformed) relative frequencies in these cells. Mixed ANOVA also tests the replicability of the results across laboratories, since the genotype difference is tested over the Genotype×Laboratory interaction as well as the within group variance. The significance is corrected for multiple comparisons using FDR as in Benjamini et al, 2001.

It is contemplated that the number of steps utilized by the current invention and the tasks performed and determinations accomplished at each step may vary without departing from the scope and spirit of the present invention.

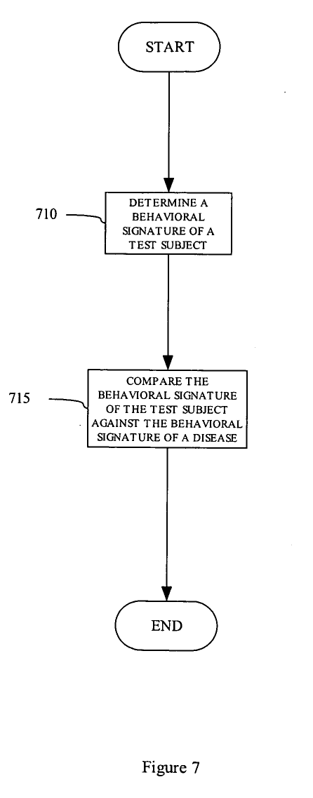

In a preferred embodiment, shown in FIG. 7, a method for diagnosing a disease utilizing the behavioral pattern analysis of the current invention is provided. The method includes a first step 710 of determining a behavioral signature of a test subject. The test subject may be thought to be afflicted with a disease or not. In a second step 715 it is determined if the behavioral signature of the test subject is consistent with a behavioral signature for a disease by comparison. In the current embodiment, the behavioral signature for the disease has been previously identified, preferably through use of the paradigm of the present invention. It is contemplated that the current method may further comprise the step of identifying the behavioral signature for the disease sing the paradigm of the current invention prior to the comparison of it with the behavioral signature of the test subject.

In the current method embodiment, the use of the current invention may allow for the early detection/diagnosis of the presence of a disease within the subject. As described below, the progression of many diseases may be exhibited through behavioral patterns. The current invention may allow for the detection of these patterns, which are undetectable using previous and/or currently available behavioral testing models. These subtle behavioral patterns may be detected at an earlier stage in the progression of the disease, thereby allowing earlier diagnosis and promoting an effective prevention/intervention and/or treatment of the disease.

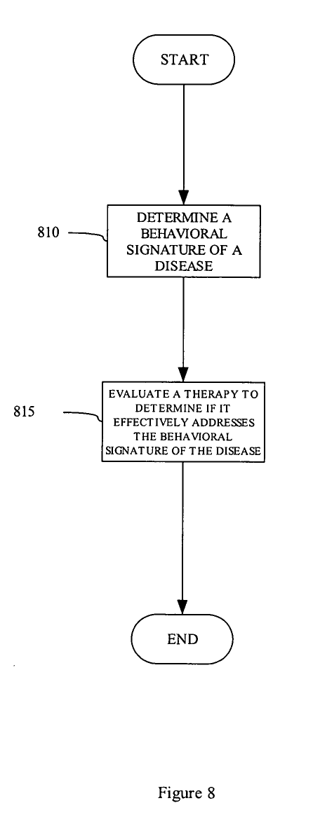

The current invention, as shown in FIG. 8, further contemplates a method of testing therapies for a disease. The method includes a first step 810 of determining the behavioral signature of a disease. From this behavioral signature a therapy may be evaluated in a second step 815 to determine if it may effectively address the behavioral patterns being exhibited. It is contemplated that the therapy may be a drug therapy or alternative form of therapy, such as a physical therapy regimen. The evaluation may further include determining the behavioral signature of the therapy and matching the therapy's behavioral signature with that of the disease. For example, in the experiment described below a certain pattern was found, which allowed for the differentiation of the movement of SOD1 rat mutants, an established model of ALS, from that of wild-type animals. The SOD1 mutants perform this pattern significantly less than the wild-type animal. If a certain therapy tested in the SOD1 mutants changes the frequency of performing this pattern back towards the value typical to the wild type animals, this will indicate that this therapy may be effective for treating ALS.

In general, the invention described above is a method for analyzing and mining behavioral data using numerous, such as >50,000, movement patterns and generating a behavioral signature from statistically significant patterns. The behavioral signature may then be utilized in various manners, such as in drug classification and drug discovery. In another preferred embodiment, the current invention may be used in diagnosing disease as is exemplified by searching for early motor symptoms in the open-field behavior of SOD1 mutant rats, an animal model of Amyotrophic Lateral Sclerosis (ALS). Through use of the current invention isolation of a unique motor pattern that differentiates the SOD1 mutants from the wild-type controls two months before disease onset was accomplished. This pattern, defined as heavy braking while moving near the arena wall but turning away from it, was performed significantly less by the SOD1 mutants when compared against the frequency of occurrence of this pattern in wild-type controls. At this early age these genotypes could not be differentiated by standard behavioral measures or subjective observation of their behavior. The discovered pattern was further validated in independent data from animals that were not used for isolating it. Such an early symptom may enable investigators to diagnose disease much earlier and/or test therapies (e.g., drug or otherwise) aimed for intervention rather than remediation. This study (described below) further demonstrates that the current invention may be used to mine complex animal behavior for subtle and reliable effects. The paradigm of the current embodiment may be readily adapted to any spatial test (as previously identified) employing automated tracking, and may prove useful in additional tests, such as the water maze, the plus maze, and photobeam activity boxes, that record large amounts of data.

Material and Methods

The data are path coordinates from the SEE open-field test (Drai, D. & Golani, I. (2001) SEE: a tool for the visualization and analysis of rodent exploratory behavior. Neurosci Biobehav Rev, 25 (5), 409-426, (hereinafter “Drai & Golani, 2001”), which is herein incorporated by reference in its entirety.) although in principle the current invention may be used with data from any spatial test, or in fact any kind of behavioral test that produces large amounts of data. SEE (Strategy for the Exploration of Exploration) is a software-based strategy, embedded in the programming environment of Mathematica™ (WOLFRAM RESEARCH, INC., Illinois), for the visualization and analysis of free spatial behavior. It was recently shown to be useful for the behavioral phenotyping of mice (Drai, D., Kafkafi, N., Benjamini, Y., Elmer, G. I. & Golani, I. (2001) Rats and mice share common ethologically relevant parameters of exploratory behavior. Behav Brain Res 125(1-2), 133-140, which is herein incorporated by reference in its entirety; Benjamini, Y., Drai, D., Elmer, G., Kafkafi, N. & Golani, I. (2001) Controlling the false discovery rate in behavior genetics research. Behav Brain Res 125(1-2), 279-284, which is herein incorporated by reference in its entirety; Kafkafi, N., Lipkind, D., Benjamini. Y., Mayo, C. L., Elmer, G. I. & Golani I. (2003a) SEE locomotor behavior test discriminates C57BL/6J and DBA/2J mouse inbred strains across laboratories and protocol conditions. Behav Neurosci 117(3), 464-477, which is herein incorporated by reference in its entirety; Kafkafi, N., Pagis, M., Lipkind, D., Mayo, C. L., Benjamini, Y., Elmer, G. I. & Golani, D. (2003b). Darting behavior: a quantitative movement pattern for discrimination and replicability in mouse locomotor behavior. Behav Brain Res 142(1-2), 193-205, (hereinafter “Kafkafi et al., 2003b”), which is herein incorporated by reference in its entirety; Lipkind, D., Sakov, A., Kafkafi, N., Elmer, G. I., Benjamini, Y. & Golani, I. (2004) New replicable anxiety-related measures of wall vs center behavior of mice in the open field. J Appl Physiol 97(1), 347-359, (hereinafter “Lipkind et al., 2004”) which is herein incorporated by reference in its entirety; Kafkafi, N., Benjamini, Y., Sakov, A., Elmer, G. I. & Golani, I. (2005) Genotype-environment interactions in mouse behavior: a way out of the problem. Proc Natl Acad Sci USA 102(12), 4619-4624, (hereinafter “Kafkafi & Benjamini et al., 2005”) which is herein incorporated by reference in its entirety; Kafkafi, N. & Elmer G. I. (2005) Texture of locomotor path: a replicable characterization of a complex behavioral phenotype. Genes Brain Behav 4(7), 431-443, (hereinafter “Kafkafi, N. & Elmer G. I., 2005”), which is herein incorporated by reference in its entirety). These studies show that, in contrast with a common view of open-field behavior as an essentially stochastic phenomenon, it is structured and consists of intrinsic behavioral building blocks. The most basic of these building blocks are “progression segments”, consisting of bouts of locomotor movement, and “lingering episodes” (“stops” in their generalized sense, consisting of both arrests and small “non-locomotor” movements). SEE employs simple properties of these building blocks and their syntax as behavioral measures (“endpoints”) for assessing open-field behavior.

Animals and Testing

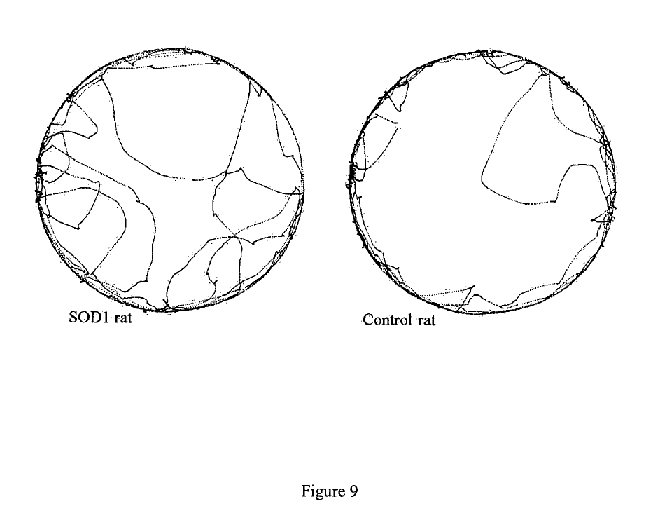

12 SOD1 mutant (G93A) and 12 Sprague-Dawley wild-type control rats, both males at 5 weeks of age, were obtained. They were housed 2-3 per cage with food and water ad libitum for two weeks on a standard dark-light cycle before the beginning of the experiment. The animals were tested at two ages: PND (post-natal day) 50-55 and PND 75-80. Each of these tests included three 30 min open-field sessions—one session per day for three consecutive days—and a grip-strength test on the fourth day. All animals were weighed before each testing time point. Open-field tests were conducted using the standard SEE procedure (Drai, D. & Golani, I., 2001; Kafkafi & Benjamini et al., 2005). Briefly, the animal is allowed to freely explore a 2.50 m diameter circular arena while its location is tracked using Noldus EthoVision® (Noldus, Netherlands) video tracking system at a rate of 30 Hz, and the {x, y, t} coordinates of the path (FIG. 9) are exported to SEE. The grip force test was conducted separately for the fore and hind legs using a metal grid connected to an isometric force transducer Columbus Instruments™ (Ohio, USA) in a procedure similar to that described by Derave, W., Van Den Bosch, L., Lemmens, G., Eijnde, B. O., Robberecht, W. & Hespel, P. (2003) Skeletal muscle properties in a transgenic mouse model for amyotrophic lateral sclerosis: effects of creatine treatment. Neurobiol Dis 13 (3), 264-72, (hereinafter “Derave et al., 2003”), which is herein incorporated by reference in its entirety: the animal was lifted by its tail and made to hold the grid with its fore or hind limbs, and then pulled backwards gently until it could no longer hold the grid. The maximal force in grams was recorded in six consecutive trials and the animal's final result was set to their median.

SEE Behavioral Procedures

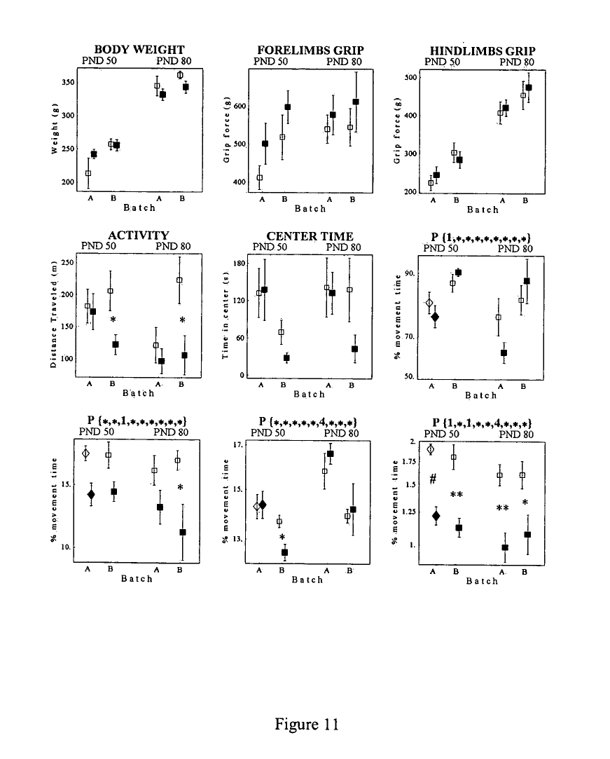

The EthoVision® (Noldus, Netherlands) path coordinates were imported into SEE (Drai & Golani, 2001), and the SEE Path Smoother procedure (Hen, I., Sakov, A., Kafkafi, N., Golani, I. & Benjamini, Y. (2004) The dynamics of spatial behavior: how can robust smoothing techniques help? J Neurosci Methods 133(1-2), 161-172, (hereinafter “Hen et al., 2004”), which is herein incorporated by reference in its entirety, which is herein incorporated by reference in its entirety) was used to filter out tracking noise. Since the animals weren't very active we pooled data from three successive sessions for each animal. At a tracking rate of 30 Hz the data file of each animal thus included 30 coordinates×60 seconds×30 minutes×3 sessions=162,000 data points. Using the usual SEE procedure the path was further divided into segments of progression and lingering (small local movements during stopping, see Drai, D., Benjamini, Y. & Golani, I. (2000) Statistical discrimination of natural modes of motion in rat exploratory behavior. J Neurosci Methods 96(2), 119-31, (hereinafter Drai et al., 2000”) which is herein incorporated by reference in its entirety; Kafkafi N, Mayo C, Drai D, Golani I, Elmer G. (2001) Natural segmentation of the locomotor behavior of drug-induced rats in a photobeam cage. J Neurosci Methods 109(2), 111-21, (hereinafter “Kafkafi et al., 2001”), which is herein incorporated by reference in its entirety). The lingering component of behavior may exhibit small spatial resolution that is difficult to reliably measured utilizing current tracking technology, therefore analysis concentrated on progression segments. Depending on the activity of the animal, the number of data points within progression segments usually consisted of 10,000-50,000 (i.e., about 5-25 minutes) per three sessions. Mutants and controls did not consistently differ in their general activity (see Results section and FIG. 11, second raw, left).

The algorithm utilized by the current invention and described in the next section was programmed in Mathematica™ (WOLFRAM RESEARCH, INC., Illinois) using the SEE package (Drai & Golani, 2001) and the SEE Experiment Explorer package (Kafkafi, N. (2003) Extending SEE for large-scale phenotyping of mouse open-field behavior. Behav Res Methods Instrum Comput 35(2), 294-301, (hereinafter “Kafkafi, 2003”), which is herein incorporated by reference in its entirety).

In typical SEE studies behavioral patterns and measures are defined one at a time (or mostly several at a time), mainly based on the experience of the investigator, using insight developed by watching the actual behavior and/or several types of graphic visualization in SEE. Once a behavioral pattern is algorithmically defined in SEE it can be tested over a database of raw path data in order to assess its ability to discriminate reliably between different genotypes and treatments. The algorithm utilized by the current invention takes this approach further by defining a whole class of (in our case)>50,000 different behavioral patterns, and screening them for those that maximize the difference between the experiment groups.

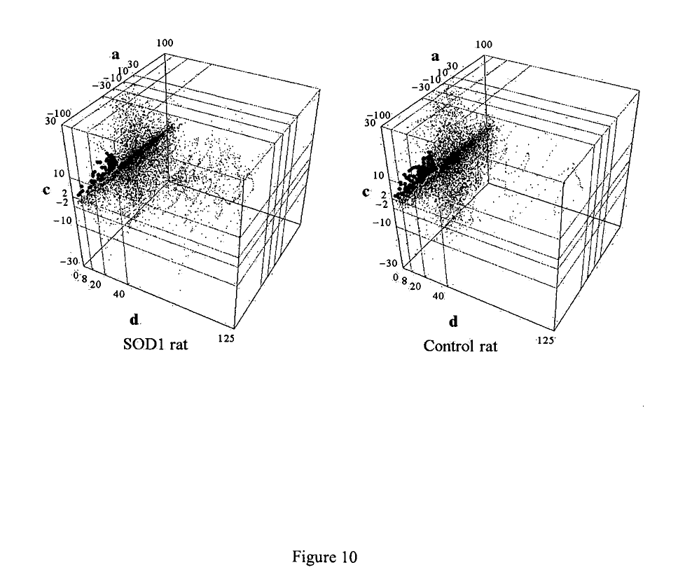

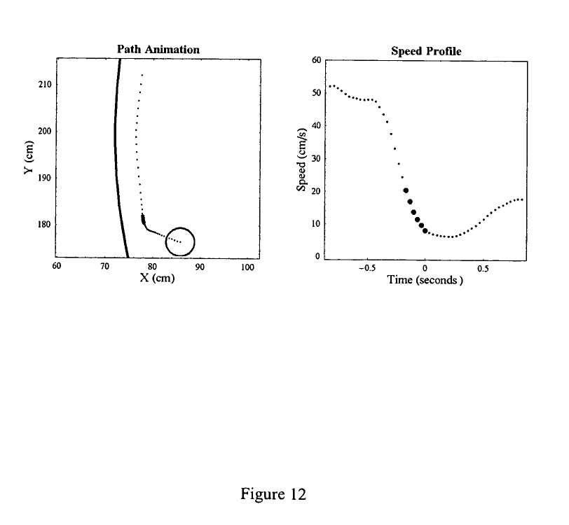

In the current embodiment, the current invention is employed in a manner similar to microarray gene chip analysis. The idea is to test a very large number of possible movement patterns in parallel, and isolate only those patterns in which a significant difference between the experiment groups is detected (in our case a difference between the SOD1 mutants and the wild-type controls, see FIG. 9). The current invention provides a paradigm to address how to construct the “virtual chip”, that is, how to dissect the behavior into many possible patterns. This is achieved by transforming each coordinate of the path into a vector of several “features” of the movement. The chosen features are dynamic variables relevant to the behavior, such as momentary speed, momentary acceleration, momentary direction of movement and momentary change of direction. The feature space is divided into many “cells” (e.g., FIG. 10), each corresponding to a specific combination of feature values, or a motor pattern (e.g., FIG. 12). The difference between the experiment and the control group is tested in the relative frequency of performing each of these behavioral patterns.

In order to address the multiplicity problem, that is, when simultaneously screening a large number of possible movement patterns we need to prohibit the occurrence of false discoveries and provide valid statistical inference for the selected patterns (Benjamini Y. & Yekutieli D. (2005) False discovery rate-adjusted multiple confidence intervals for selected parameters. J Amer Stat Assoc 100, 71, (hereinafter “Benjamini & Yekutieli, 2005”), which is herein incorporated by reference in its entirety), the current invention may employ the Bonferroni Criterion for screening the significant movement patterns—using a corrected significance level of 0.05/n, where n is the number of potential movement patterns, thus ensuring that the probability of discovering even a single false movement pattern is less than 0.05. To further ensure the validity of the inference for the selected movement patterns the animals may be divided into independent “mining set” and “test set”. The mining set may be used for isolating the best patterns as described above, while the statistical inference for the selected movement patterns may be based on the independently distributed test set. The following description provides an exemplary step-by-step algorithmic progression employed in preferred embodiments of the present invention.

Input: The inputs for the algorithm are the (t, x, y) coordinates of the animal's path in the arena belonging only to progression segments (see Methods section and FIG. 9. More details in Drai et al, 2000; Kafkafi et al., 2001) measured at a rate of 30 Hz. Progression segments are typically 6-300 data points in length (i.e., 0.2-10 seconds in duration) and a session typically includes several hundreds of them to a total of 10,000-50,000 data points per animal.

Step 1: Each data point is quantified using an m-dimensional vector of “features” of movement. In this study we used m=9 features which are defined in Table 1. These features, and others or alternative features, may be employed by the current invention as relevant to open-field behavior. The distance from the wall d was shown to measure heritable thigmotactic behavior (e.g., Broadhurst, P. L. (1975). The Maudsley reactive and nonreactive strains of rats: A survey. Behav Genet 5, 299-319, (hereinafter “Broadhurst, 1975”), which is herein incorporated by reference in its entirety; Ramos, A., Berton, O., Mormede, P. & Chaouloff, F. (1997) A multiple-test study of anxiety-related behaviours in six inbred rat strains. Behav Brain Res 85(1), 57-69, (hereinafter “Ramos et al., 1997), which is herein incorporated by reference in its entirety; Leppanen, P. K., Ewalds-Kvist, S. B. & Selander, R. K. (2005) Mice selectively bred for open-field thigmotaxis: life span and stability of the selection trait. J Gen Psychol 132(2), 187-204, (hereinafter “Leppanen et al., 2005”), which is herein incorporated by reference in its entirety; Lipkind et al., 2004). The momentary speed v was shown to be a key variable in the intrinsic categorization of behavior into progression and “lingering” in both mice (Drai et al., 2000) and rats (Kafkafi et al., 2001). The acceleration a was shown to have high heritability and reliability in mice (Kafkafi et al., 2003b). The jerkj is the derivative of acceleration according to time, or the second derivative of the speed. It was chosen because speed peaks were shown to be a meaningful component of rodent behavior (Drai et al, 2000; Kafkafi et al., 2001) and the jerk is required to distinguish between speed peaks (near-zero acceleration and negative jerk) and local minima of speed (near-zero acceleration and positive jerk). The momentary heading h (direction of movement relative to the arena wall) may be an important aspect of open-field behavior in mice. The path curvature in a scale of 4 cm and 16 cm (c₄ and c₁₆ respectively) were shown to discriminate several genotypes of mice with high heritability and reliability (Kafkafi, N. & Elmer G. I. (2005) Texture of locomotor path: a replicable characterization of a complex behavioral phenotype. Genes Brain Behav 4(7), 431-443, (hereinafter “Kafkafi & Elmer, 2005), which is herein incorporated by reference in its entirety). Finally, features tₛ and tₑ quantify the temporal location of the data point within progression segments (Drai et al., 2000; Kafkafi et al., 2001), thus making it possible to mine patterns that always take place in identifiable “time periods” of various progression segments, e.g., the beginning or end of progression segments.

The path of the animal in the arena (FIG. 9) is thus transformed into a collection of trajectories (progression segments), each typically including several tens of data points, each data point consisting of a 9-dimensional vector of the form (d, V, a, j, h, c₄, c₁₆, tₛ, tₑ), shown in Table 2, in the feature space (FIG. 10).

TABLE 2
The 9 behavioral features

| | | | Number of | |
|---|---|---|---|---|
| Symbol | Feature Definition | Units | Intervals | Interval Edges |
| d | Momentary distance from arena wall | cm | 4 | 0, 8, 20, 40, 125 |
| v | Momentary speed of movement | cm/s | 4 | 0, 20, 40, 60, >60 |
| a | Momentary acceleration of movement | cm/s² | 5 | <−30, −30, −5, 5, 30, >30 |
| j | Momentary jerk (change in acceleration) of movement | cm/s³ | 5 | <−300, −300, −50, 50, 300, >300 |
| h | Momentary movement direction relative to wall (heading) | degrees | 5 | −90, −30, −5, 5, 30, 90 |
| c₄ | Momentary path curvature in a 4 cm scale | degrees/cm | 5 | <−10, −10, −2, 2, 10, >10 |
| c₁₆ | Momentary path curvature in a 16 cm scale | degrees/cm | 5 | <−5, −5, −1, 1, 5, >5 |
| tₛ | Time from start of progression segment | s | 3 | 0, 0.2, 1, >3 |
| tₑ | Time to end of progression segment | s | 3 | 0, 0.2, 1, >3 |

d: Momentary distance from arena wall: This is a well-established feature of rodent open-field behavior (e.g., Ramos et al., 1997). It is generally thought to measure wall-hugging or thigmotaxis behavior, which is usually related to “anxiety” and “emotionality”. Both rats (Broadhurst, 1975) and mice (Leppanen et al, 2005) have been selected for increased and decreased thigmotaxis. In mice, the distance from the wall was shown to be a factor in the intrinsic organization of the behavior (Lipkind et al., 2004), and the wall has a strong effect on the direction of movement even when the animal is at distance from it. (Horev, G., Benjamini, Y., Sakov, A. & Golani, I. (in press) Estimating wall guidance and attraction in mouse free locomotor behavior. Genes Brain Behav, which is herein incorporated by reference in its entirety).

Since the animals tend to stay much more near the wall, 4 intervals of increasing distance were defined: 0 to 8 cm from the wall (approximately the range of maintaining a physical contact with the wall), 8 to 20 cm from the wall close proximity but not physical contact, 20 to 40 cm (slightly away from the wall), 40 to 125 cm (far away from the wall).

v: Momentary speed of movement: the speed was shown to be a key variable in the intrinsic categorization of behavior to progression and “lingering” in both mice (Drai et al., 2000) and rats (Kafkafi et al., 2001). 4 intervals of speed were defined: 0 to 20 cm/s (slow), 20 to 40 cm/s (medium), 40 to 60 cm/s (fast) and above 60 cm/s (very fast). The speed was computed and noise filtered using the LOESS (hereinafter “LOWESS”) algorithm as described in Hen et al., 2004, with a moving window width of 0.4 s.

a: Momentary acceleration of movement: Acceleration was shown to be a key variable discriminating the behavior of different genotypes of mice, reliably and with high broad-sense heritability (Kafkafi, 2003). 5 unequal intervals of acceleration that produce approximately similar frequency were defined: less than −30 cm/s (strong deceleration, meaning heavy braking), −30 to −5 cm/s (mild deceleration), −5 to 5 cm/s (approximately uniform speed) 5 cm/s to 30 cm/s (mild acceleration), more than 30 cm/s (high acceleration). The acceleration was computed and noise filtered using the LOWESS algorithm as described in Hen et al., 2004, with a moving window width of 0.4 s.

j: Momentary jerk of movement: Jerk is the derivative of acceleration according to time, or the second derivative of the speed. This feature is relevant since speed peaks have been shown to be a meaningful component of rodent behavior (Drai et al., 2000; Kafkafi et al., 2001) and the jerk is required to distinguish between speed peaks (near-zero acceleration and negative jerk) and local minima of speed (near-zero acceleration and positive jerk). 5 unequal intervals of jerk that produce approximately similar frequency were defined: less than −300 cm/s (very negative jerk, meaning a strong decrease in acceleration), −300 to −50 cm/s (mild decrease in acceleration), −50 to 50 cm/s (approximately uniform acceleration), 50 cm/s to 300 cm/s (mild increase in acceleration), more than 300 cm/s (strong increase in acceleration). The jerk was computed and noise filtered using the LOWESS algorithm as described in Hen et al., 2004, with a moving window width of 0.4 s.

h: Momentary movement direction (“heading”) relative to wall: Horev at al., (in press) showed the effect of the wall on heading even from distance. The heading in degrees relative to the arena wall was determined, with negative values representing movement towards the wall and positive values away from it. 5 unequal intervals of heading that produce approximately similar frequencies were defined: −90° to −30° (moving in the towards the wall), −30° to −5° (moving slightly towards the wall), −5° to 5° (moving approximately parallel to the wall), −90° to −30° (moving slightly away of the wall), 30° to 90° (moving away of the wall). The determined direction and noise were filtered using the LOWESS algorithm as described in Hen et al., 2004, with a moving window width of 0.4 s.

c₄: Path curvature in a 4 cm scale: This feature measures the momentary turning (change of direction) in a unit of path length. Kafkafi & Benjamini et al., 2005, shows that the curvature of the path has high heritability in the mouse and can be use to differentiate inbred strains with high replicability across laboratories. This study also showed that the curvature measured in a 4 cm scale (smaller than the animal body) is not necessarily correlated with the curvature measured in a 16 cm scale (approximately body length in rats). Thus, the curvature was used in both scales as features in this study. The curvature in 64 cm scale, also used in the above study, was not used because only a small portion of the segments were longer than 64 cm. Curvature was computed as detailed in Kafkafi & Elmer, 2005, except for one difference: rather than using the sign to differentiate between left and right we used it here to differentiate between the direction towards the arena wall or away from it. As in h, negative curvature values indicate turning towards the wall and positive curvature values indicate turning in the direction away from the wall. 5 unequal intervals of curvature that produced approximately similar frequencies were defined: less than −10 degree/cm (turning sharply towards the wall), −10 to −2 degree/cm (turning slightly towards the wall), −2 to 2 degree/cm (moving approximately straight ahead), −2 to −10 degree/cm (turning slightly away of the wall), more than 10 degree/cm (turning sharply away of the wall). Note that the curvature was computed relative to a distance rather than time unit (because calculating it over very small distances is very sensitive to measurement error), meaning it represent different time windows depending on the speed, e,g, in a typical speed of 16 cm/s using 4 cm scale implies a time window of 4/16 or 0.25 seconds.

c₁₆: Path curvature in a 16 cm scale: See the previous feature for properties of path curvature and computing it in different distance scales. 5 unequal intervals of curvature that produced approximately similar frequencies were defined: less than −5 degree/cm (turning sharply towards the wall), −5 to −1 degree/cm (turning slightly towards the wall), −1 to 1 degree/cm (moving approximately straight ahead), −1 to −5 degree/cm (turning slightly away of the wall), more than 10 degree/cm (turning sharply away of the wall). Note that the curvature was computed relative to a distance rather than time unit (because calculating it over very small distances is very sensitive to measurement error), meaning it represent different time windows depending on the speed, e,g, in a typical speed of 16 cm/s using 4 cm scale implies a time window of 4/16 or 0.25 seconds.

tₛ: time for start ofprogression segment: Progression segments were shown to be a primary natural primitive of rodent spatial behavior (Drai et al., 2000; Kafkafi et al., 2001). Only data points belonging to progression segments were used in this study design. By definition, a progression movement starts and ends with complete immobility. Certain movement patterns may be affected if they take place immediately after the beginning of the segment, immediately before it ends, or anywhere in the middle. 3 unequal intervals of time from the start of the segment were defined: less than 0.2 s (6 data points in our 30 Hz measurement rate), 0.2 to 1.0 s (30 data points) and more than 1.0 s.

tₑ: time to end of progression segment: A similar rationale as that shown for tS above forms the basis for the use of this feature. 3 unequal intervals were defined: less than 0.2 s, 0.2 to 1.0 s (30 data points) and more than 1.0 s.

Step 2: The range of each feature is partitioned into several disjointed intervals, thus dividing the feature space into many “cells” (grid lines in FIG. 10). Table 2 (above) shows the number of intervals for each feature and the values chosen for the interval edges. Note that the intervals may vary and may not be of equal length. In the current embodiment, the intervals promote the definition of cells of approximately equal frequencies (see below). For example, rats and mice typically move near the wall much more frequently than in the center of the arena, and therefore the distance from the wall d was divided into boundaries of 0, 8, 20, 40 and 125 cm (note the horizontal axes in FIG. 10).

Step 3: Dividing the feature space using all m=9 dimensions may result in a huge number of overly-specified cells, most of them including too few data points for a significant sample size and highly vulnerable to random variation. In this study we thus limit ourselves to all the feature subspaces up to 4 dimensions. For example, FIG. 10 (2) shows the 3-dimensional subspace including the three features d, a and c₄. Each cell is denoted by an i.d. of the form P{i₁, i₂, i₃, . . . , iₘ} corresponding to the m-dimensional feature vector, P representing the pattern (behavioral pattern) be defined, where i is the index of the interval in the corresponding feature according to Table 2, and an asterisk denoting a feature that is not relevant for the definition of the cell. For example, P{1,*,1,*,*,4,*,*,*} denotes a cell in which values of the 1st feature belong to the 1st interval in that feature, values of the 3rd feature belong to the 1st interval in that feature, values of the 6th feature belong to the 4th interval in that feature, and the other features (asterisks) are irrelevant to the definition of the pattern and can accept any value. Limiting the algorithm to four relevant features at most means that 50,674 cells having at least 5 asterisks are considered.

Step 4: In each cell we consider the relative frequency of data points falling into this cell, using the Logit transformation:

$$( Eq . 1 \text{:} )$$

$$LogitFrequency ( P \{ {i}_{1} , {i}_{2} , {i}_{3} , … , {i}_{m} \} ) = log ( \frac{k + 1 / 3}{l - k + 1 / 3} )$$

where k is the number of data points falling in this cell and l is the total number of data points for this animal (see FIG. 10). The Logit transform is routinely used in statistics (e.g. logistic regression) to transform proportions bounded between 0 and 1 to real valued variables more amenable to statistical analysis, and adding ⅓ is useful for correcting the behavior of the transformation when k=0.

Step 5: Cells with very small support—feature combinations that were hardly exhibited by most or all animals may be considered irrelevant and were discarded in the current embodiment. Many combinations of feature values are rarely used due to trivial physical limitations on movement (e.g., accelerating during a sharp turn at high speed). Other combinations are simply things that rats in general prefer to avoid (e.g., running towards the center of the arena at high speed and near-zero acceleration). Such physical and behavioral limitations, however, may differ across the experiment groups, and the avoidance of discarding a cell that generally has a low frequency if it is frequent in one of the groups is promoted by the current invention. Therefore we compute the median LogitFrequency in each group of mining set samples and discard the cell only if the maximal group median (whatever group it is) is lower than FrequencyCutoff. In this study FrequencyCutoff was set to −5.5, which corresponds to 60 data points (i.e., 2 cumulative seconds) or using this pattern for about 0.4% of the total progression time in an animal with typical activity of I=15,000 data points. After this step we are thus left with B_{non-neg}—the set of non-discarded movement patterns.

Step 6: Discover the movement patterns in B_{non-neg} differing in relative frequency in the two experimental groups. In this study we apply a two sample t-test to compare the mining set mean LogitFrequency values between the SOD1 animals and the wild-type controls, and screen the subset of potentially significant movement patterns B_{pot-sig}⊂B_{non-neg} using the Bonferroni criterion. That is, we test each null hypothesis at level α/n where n is the number of comparisons (i.e., the number of cells in B_{non-neg}) at a level of α=0.05.

Step 6a (optional): Within the remaining potentially-significant patterns B_{pot-sig} a high level of cross-pattern correlation may exist, especially since some of these patterns overlap in their definition. In this case it is possible to use a variety of procedures to screen these patterns further in a way that reduces cross-correlation. However, in our case the objective was to find at least one pattern that discriminates the mutant SOD1 animals from the control animals, and generally their behavior is so similar that very few differences, if at all, are likely to be found. We thus simply picked the most significant pattern out of B_{pot-sig}.

Step 7: The test set samples ma be used to validate the discrimination ability of the movement patterns discovered in the mining set. According to Benjamini & Yekutieli, 2005, the test set inference may be corrected for multiplicity for the B_{pot-sig} screened patterns. If no patterns are found significant in the mining set it may be possible to add the data from the test set to the mining set in order to increase the sample size and hopefully detect a significant pattern in step 6, at the cost of leaving no data for cross-validation in step 7.

Applying the current invention's use of the algorithm in this study we divided the animals into two batches A (7 mutants vs. 7 controls) and B (5 mutants vs. 5 controls). Batch A at PND 50 was used as the mining set, and the isolated pattern was tested in batch B at PND 50 and in both batches A and B at PND 80. One very inactive control animal in batch A had to be discarded from this Pattern Array analysis, both in PND 50 and 80, but it was still considered for the analysis of body weight, grip force, activity and center time (FIG. 11).

In step 5, out of the total 50,674 behavioral patterns, 11,831 patterns were found common enough in the mining set to pass FrequencyCutoff. The Bonferroni Criterion at a=0.05 was thus set to 0.05/11831=4.226×10⁻⁶. Out of these patterns only two were found to be more significant than this criterion. The more significant of the two (p=2.9×10⁻⁶) was P{1,*,1,*,*,4,*,*,*}. This i.d. vector shows that six out of the nine features were irrelevant for this pattern and may take any value (asterisks). Of the others, the first feature refers to the distance from the arena wall d, and the index of 1 in this place (see Table 1) denotes the lowest level of this feature, which is less than 8 cm from the wall. The third feature refers to the acceleration a, and 1 denotes the most negative acceleration level, actually a strong deceleration (braking). The sixth feature c₄ refers to path curvature (change of direction) in a scale of 4 cm (Kafkafi & Elmer, 2005), and the index 4 denotes a slight turn in the direction away from the arena wall. Thus P{1,*,1,*,*,4,*,*,*} is defined as braking strongly while moving very close to the wall but turning slightly away from it. An actual example of a rat performing this pattern can be seen in FIG. 12. At PND 50 the wild-type controls performed this pattern on average for about 1.8% of their progression time (FIG. 11, bottom right), while the SOD1 animals performed it on average for only 1.2% of their progression time. This pattern was then tested for significance in the test sets.

FIG. 11 shows the results in body weight, grip strength of forelimbs and hindlimbs, and the open-field behavior using six measures: the widely-used activity and center time, and four patterns. These are the discovered pattern P{1,*,1,*,*,4,*,*,*} and the three single-feature patterns that intersect to generate it P{1,*,*,*,*,*,*,*,*}, P{*,*,1,*,*,*,*,*,*} and P{*,*,*,*,*,4,*,*,*}. Body weight and grip strength of either forelimbs or hindlimbs have increased as expected from PND 50 to PND 80, but there was no significant difference between mutants and controls. In batch B the mutants were significantly less active, but this difference was not replicated in batch A at both time points. Patterns P{1,*,*,*,*,*,*,*,*} (i.e., moving near the wall) also failed in significantly differentiating the SOD1 rats from the controls. Patterns P{*,*,*,*,*,4,*,*,*} (i.e., turning slightly away from the wall) and P{*,*,1,*,*,*,*,*,*} (i.e., braking strongly) just barely passed significance in one comparison, but not in the other three. That is, each of the isolated features did not differentiate the SOD1 mutants by itself. Their intersection, however, the screened pattern P{1,*,1,*,*,4,*,*,*}, consistently differentiated the two groups, with the SOD1 mutants always performing it significantly less than the wild-type controls. Note that the small variability in batch A at age 50 days (diamonds instead of squares) might be misleading, since these data were the “mining set” used for the very discovery of this pattern, and by definition it was the most significant out of the 50,674 tested patterns. However, this pattern was also significant in the test sets: batch B at 50 days of age (t=4.5; p<0.01; n=5, 5), batch A at 80 days (t=4.0; p<0.01; n=7,7) and batch B at 80 days (t=2.5; p<0.05; n=5, 5, all using t-test). Since here we only consider a single pattern there is no need to correct the test set results for multiplicity. Note that batch A in PND 50 and PND 80 are the same animals, and therefore the second is not independent of the first. However, batch B is an independent validation of batch A in both PND 50 and 80. Note also that batch B not only replicated the differences discovered in batch A, but the absolute frequencies of performing the pattern were very similar.

SOD1 mutant animals are generally considered presymptomatic before the age of 80 days old in mice (Chiu, A. Y., Zhai, P., Dal Canto, M. C., Peters, T. M., Kwon, Y. W., Prattis, S. M. & Gurney, M. E., (1995) Age-dependent penetrance of disease in a transgenic mouse model of familial amyotrophic lateral sclerosis. Mol Cell Neurosci 6, 349-362, which is herein incorporated by reference in its entirety; Weydt, P., Hong, S. Y., Kliot, M. and Moller, T. (2003) Assessing disease onset and progression in the SOD1 mouse model of ALS. Neuroreport 14 (7), 1051-4, which is herein incorporated by reference in its entirety; Derave et al., 2003) and 90 days old in rats (Matsumoto, A., Okada, Y., Nakamichi, M., Nakamura, M., Toyama, Y., Sobue, G., Nagai, M., Aoki, M., Itoyama, Y. & Okano, H. (2006) Disease progression of human SOD1 (G93A) transgenic ALS model rats. J Neurosci Res 83 (1), 119-33, (hereinafter “Matsumoto et al., 2006”), which is herein incorporated by reference in its entirety). The earliest behavioral symptom reported in SOD1 animals is decreased performance on the accelerating rotarod at PND 78 in SOD1 mice (Fischer, L. R., Culver, D. G., Tennant, P., Davis, A. A., Wang, M., Castellano-Sanchez, A., Khan, J., Polak, M. A. & Glass, J. D. (2004) Amyotrophic lateral sclerosis is a distal axonopathy: evidence in mice and man. Exp Neurol 185, 232-240, (hereinafter “Fischer et al., 2004”), which is herein incorporated by reference in its entirety). Indeed, using the grip strength, the common measure of disease onset in the SOD1 animals, we could not detect a significant effect of the mutation in our rats at either 50 or 80 days of age (FIG. 11). Furthermore, in agreement with a recent extensive report (Matsumoto et al., 2006) and our own past experience, before PND 90 no difference between the SOD1 rats and the wild-type controls could be detected from subjective observation of their open-field behavior or any other aspect of behavior.

In contrast, the method of the current invention was able to discover a movement pattern that significantly and consistently differentiated the SOD1 rats at PND 50 and 80 in comparatively small group sizes (5-7 animals). This difference may be related to the denervation found in the gastrocnemius, soleus, and tibialis anterior muscles of SOD1 mice, which included 40% of end-plates by PND 47 and continued to progress up to the time of death (Fisher et al., 2004). The discovered premorbid symptom may enable investigators to test treatments for delaying or even preventing the disease.

Performance of the isolated pattern slightly decreased in both mutants and controls as a function of age (FIG. 11, bottom right). This is reasonable since they were heavier by PND 80, which makes strong braking more difficult. The pattern did not detect any increase of the difference between mutants and controls from PND 50 to PND 80, which would be expected if early symptoms were getting worse as the mutants approach the age of disease onset. In the current embodiment, the patterns are mined based on t-test comparison between the experiment and control group in one case (the PND 50 mining set). It is contemplated that the mining may occur using various and alternative patterns and be based on various other tests to provide comparison of data. A slightly more advanced application may test a mining set along several ages using, e.g., two-way ANOVA of Genotype×Age with Age as a repeated measure. Such a mining set may discover another pattern that more specifically tracts disease progression. Many kinds of statistical tests may be used in the current invention, (e.g., t-test, one-way ANOVA, two-way ANOVA with fixed or mixed model, ANOVA with repeated measures, linear and non-linear regression tests, and generally any statistical test producing a p-value) depending on the design of the data and the objective of testing.

While it is not clear yet why this specific pattern is less performed by the mutants, the methodology of the current invention may still be used to explore the results of similar patterns in order to gain some insight regarding the important characteristics, as FIG. 11 demonstrates with the three single-feature patterns. They suggest that the mutants are generally deficient in strong braking (i.e., P{*,*,1,*,*,*,*,*,*}) but not in the other two components, moving near the wall and the slight turn. These components merely interact with the braking to make the difference more pronounced and consistent. It is of course possible to explore the results in any number of additional patterns, although multiplicity considerations should to be addressed in such case.

The SOD1 mutant rat was discussed in this study as a typical case of an animal model in which the standard behavioral tests fail to detect some desirable effect (for other typical examples see Grammer, M., Kuchay, S, Chishti, A. & Baudry, M. (2005) Lack of phenotype for LTP and fear conditioning learning in calpain 1 knock-out mice. Neurobiol Learn Mem 84(3), 222-227, which is herein incorporated by reference in its entirety; Perez, F. A. & Palmiter, R. D. (2005) Parkin-deficient mice are not a robust model of parkinsonism. Proc Natl Acad Sci USA 102(6), 2174-2179, which is herein incorporated by reference in its entirety). Even when significant behavioral effects are discovered they might prove difficult to replicate in different laboratories or in slightly different conditions (Crabbe, J. C., Wahlsten, D. & Dudek, B. C. (1999) Genetics of mouse behavior: Interactions with laboratory environment. Science 284 (5420), 1670-1672, which is herein incorporated by reference in its entirety; Chesler, E. J., Wilson, S. G., Lariviere, W. R., Rodríguez-Zas, S. L. & Mogil, J. S. (2002) Influences of laboratory environment on behavior. Nature Neurosci 5, 1101-1102, which is herein incorporated by reference in its entirety; Wahlsten, D., Rustay, N. R., Metten, P. & Crabbe, J. C. (2003) In search of a better mouse test. Trends Neurosci 26(3), 132-136, which is herein incorporated by reference in its entirety; Kafkafi & Benjamini et al., 2005). The discovery of reliable behavioral endpoints with predictive validity, even without a clear understanding of their etiology, may significantly improve intervention research (Willner P (1991) Methods for assessing the validity of animal models of human psychopathology. In: Animal models in psychiatry, I, 18 Edition (Boulton A A, Baker G B, Martin-Iversen M T, eds), pp 1-24. New Jersey: Humana Press, which is herein incorporated by reference in its entirety). In such cases the ability of the current invention to test tens of thousands of hypotheses in parallel is likely to prove more powerful. While demonstrated here in open-field data, the application of the current invention may be adapted in a relatively straightforward manner to other spatial tests employing automated tracking, by choosing an appropriate set of features, and may prove useful in additional tests that record large amounts of raw data. In some cases such data may have already been measured and stored, but was not used except for computing a small number of traditional behavioral endpoints.

By testing a large number of combinations in parallel the current invention may significantly enhance the ability to propose a feature variable that would detect an effect, especially in complex patterns that may be difficult to guess from the outset. The use of a strict multiple comparisons criterion in the mining set and validating in a test set ensures that the parameters were not selected to discover circumstantial differences in the particular data set.

The current invention may not be limited to treating the data as a time series and in the preferred embodiment described above it makes use of dynamical features such as momentary speed and acceleration. In the current embodiment, the features in this study all have a short time scale (mostly estimated with a window size of about a second or less) and therefore are designed to detect brief behavior patterns of the kind that is usually associated with motor symptoms. These features are unlikely to detect more prolonged patterns that are characterized by certain syntax of basic motor building blocks, and are usually associated with more cognitive functions. In principal

In the preferred embodiments described above, the time scale used for the measurement of the features has been short, one second or less. The current invention contemplates the use of features designed for longer time scales, more than one second, allowing the detection of more prolonged patterns that may be characterized by certain syntax of basic motor building blocks, and are usually associated with more cognitive functions. It may be found that for a given session duration this will decrease the number of data points, and consequently the power to detect an effect. The current invention allows for various configurations to be employed in designing the feature space in term of endpoints and endpoint parameters in order to discover behavioral patterns specific to unique environments, test conditions or cognitive domains. As an example, the time domain within each feature may be expanded to search for behavioral patterns that occur over a larger time window, thus enabling exploration of sequential patterns that may reflect memory intensive aspects of behavior or it may be shortened to search for behavioral patterns that are predominantly motor or reflexive in nature.

The present invention is described using a particular number of features and a particular number of intervals in each feature, which together determine the total number of patterns to be tested. It is to be understood that too few patterns will decrease the chance that one of them is the most appropriate pattern, while too many patterns will result in an overly restrictive multiple comparisons criterion, which might result in failing to identify a significant difference in a pattern even if it was tested. Increasing the number of animals and the number of data points per animal may generally increase the level of significance of true positives, thus increasing the number of patterns that may be tested and the chance to pinpoint on the most appropriate pattern. The testing of many patterns is likely to increase power and flexibility relative to standard behavioral tests.

The current invention is applicable to animal models in which even a single discovered effect with predictive validity may be of importance and may be especially beneficial with behavioral data because the relevant variables in behavior are frequently not well understood. Moreover, the current invention may overcome some of the problems associated with the standard behavioral tests wherein because of the complexity of most behavioral phenomena, failing to define the variable in precisely the proper way may result in a failure to detect any effect by standard testing. Thus, instead of looking for an effect using a few hardwired variables, the current invention provides the capability to sacrifice part of the data as a mining set for isolating better variables, hence increasing the chance of finding an effect in the remaining test set.

The current invention method fits well into the approach proposed by Kafkafi & Benjamini et al., 2005, of keeping databases of raw behavioral data from many experiments, treatments and laboratories. Once a new pattern was detected in one experiment using the current invention this pattern may be immediately tested over the whole database, thus gaining insight into its meaning, consistency and generality. In such a strategy the data from each experiment may be useful beyond merely confirming or rejecting the original hypothesis.

It is to be understood that the methodology of the current invention for the diagnosis of disease onset and testing of therapy effectiveness may be used in a similar manner for the methodology outlined for the determination of a behavioral signature for drug classification and drug discovery previously described.

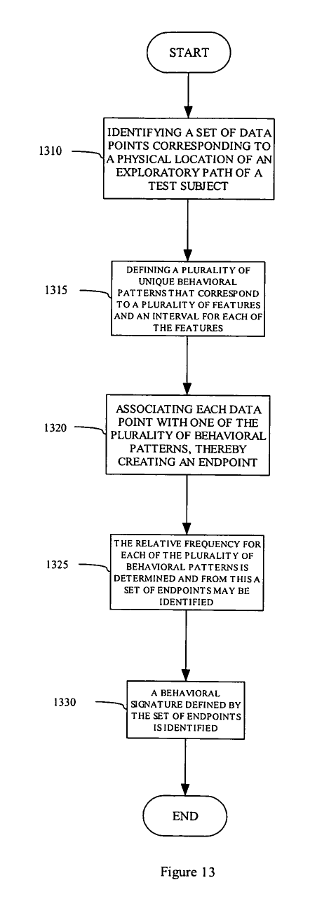

In a preferred embodiment, the current invention provides a method for determining a behavioral signature which may be employed for drug classification, drug discovery, disease diagnosis, therapy testing and/or various other models as contemplated. As shown in FIG. 13, a first step 1310 includes the identifying of a set of data points corresponding to a physical location of an exploratory path of a test subject. From this identification a plurality of unique behavioral patterns may be defined that correspond to a plurality of features and an interval for each of the features in a second step 1315. In step 1320 each data point may be associated with one of the plurality of behavioral patterns, thereby creating an endpoint. In step 1325 the relative frequency for each of the plurality of behavioral patterns is determined and from this a set of endpoints may be identified. Finally, in step 1330, a behavioral signature defined by the set of endpoints is identified.

As stated previously, the paradigm of the current invention may be utilized across various test models and applied to various data sets. These data sets may be identified as having been collected as part of the method of the invention or as having been gathered from a storage of data set information collected from previous work. The statistical significance may vary amongst different applications of the methodology of the current invention in order to allow for the creation of an appropriate model from which useful behavioral information may be gathered. The techniques used to determine statistical significance and/or correlation variance may be similar to those described above or vary to include other techniques well known to those of ordinary skill in the art.

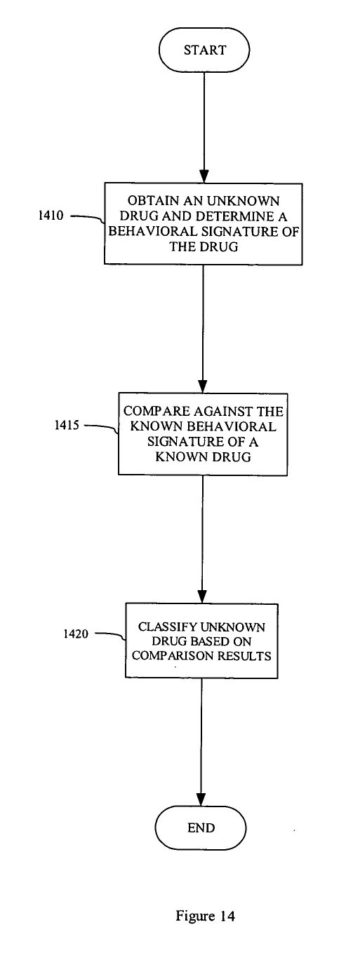

In a preferred embodiment of the present invention, shown in FIG. 14, a method of drug discovery is provided including the step 1410 of obtaining an unknown drug and determining the behavioral signature of the unknown drug. The behavioral signature may be obtained through use of the methodology of the current invention described herein. Upon determining the behavioral signature of the unknown drug, in step 1415 it is compared against the behavioral signature of a known drug. The behavioral signature of the known drug may be taken from the repository mentioned above. It is contemplated that the repository may store information related to each of the drugs in addition to the behavioral signature information. This additional information may be used in the final step 1420 of the current method wherein the unknown drug is classified based upon a significant correlation between the behavioral signature of the unknown drug and the behavioral signature of the known drug. The significance of the correlation may be established as a p-value or various other measures as may be contemplated by those of ordinary skill in the art. It is further contemplated that the level of significance may vary based upon numerous factors, such as the behavioral signature or other information of the known drug, various other information known about the unknown drug, or use of various different correlation techniques which may be employed.

Applying the same behavioral signature identification methodology proposed above, it is another preferred embodiment of the current invention to provide a system for characterizing, including the identification of the effects of the drug and classifying its behavioral signature (psychopharmacological profile), novel psychoactive drugs through comparison against known behavioral signatures (psychopharmacological profiles) of known drugs. In the current embodiment, the system includes a repository (library or database) of data, including the psychopharmacological profiles of a plurality of drugs. This repository may allow access to the data in various manners, such as manual or automated searching. The system further comprises a comprises a computer that is communicatively coupled with the repository and capable of processing, from the input of a novel psychopharmacological profile of a novel drug, the performance of a comparison of the novel drug's profile against the profiles stored in the repository. The computer and repository being capable of allowing the searching of the database, identification of relevant profiles and comparison against the novel profile.

The repository (database) of information regarding the characterization of a broad range of drugs may be created in a variety of ways. The use of such information may be employed with any of the novel embodiments described herein and others as may be contemplated by those of skill in the art.

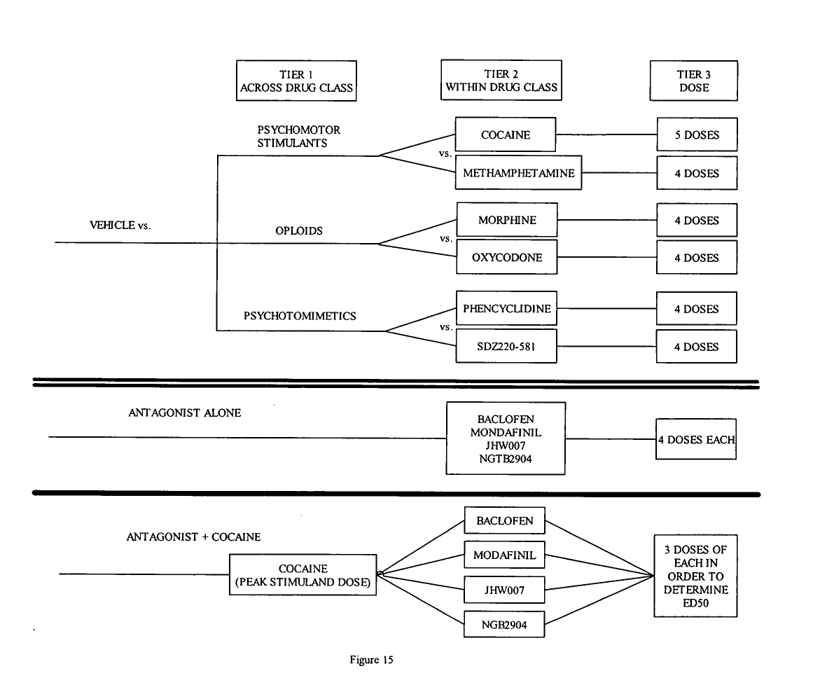

By way of example, the current invention may create a database of the unique psychopharmacological profiles of a range of psychoactive compounds. This may be realized in the context of three tiers, each tier more demanding than the previous. It is contemplated that the number and classification of the tiers may vary without departing from the scope and spirit of the present invention. In the current embodiment, the three tier characterization schema is a systematic approach characterizing a drug and is outlined in FIG. 15. A first tier may discriminate drugs across drug class. Drugs representative of major psychoactive drug classes (psychomotor stimulants, psychotomimetics and opioids) are characterized. A second tier may discriminate drugs within a drug class. Drugs within the same drug class that are structurally dissimilar or are known to have different psychoactive profiles are characterized. A third tier may discriminate based upon dose within a single drug. Each tier provides information both in terms of the psychopharmacological properties of the compounds and the strengths and weaknesses of the model.

For the characterization and identification of possible therapeutics, the current invention contemplates, determining the algorithmically derived ‘pattern profile’ of potential cocaine therapeutics and then determining the ‘pattern profile’ when given in combination with cocaine. Cocaine therapeutics currently in clinical trials (baclofen, modafinil) as well as novel therapeutics such as the D3 antagonist (NGB2904) and benzotropine analogue (JHW007) are analyzed. This characterization of the therapeutics may provide a behavioral fingerprint of novel compounds with potential clinical efficacy and provide a template for use in screening novel compounds.

Research Design and Method

The drugs may be chosen based upon several criteria. By way of example, in a preferred embodiment, shown in Table 3 (below), the first tier the chosen drugs are representative of major psychoactive drug classes with abuse liability; psychomotor stimulants, opioids and psychotomimetics. In the second tier, the chosen drugs are in the same drug class but are structurally dissimilar or known to have different pharmacological profiles. For example, methamphetamine differs from cocaine in its dopamine transporter effects (Pifl, C., H. Drobny, H. Reither, O. Hornykiewicz, and E. A. Singer, Mechanism of the dopamine-releasing actions of amphetamine and cocaine: plasmalemmal dopamine transporter versus vesicular monoamine transporter. Mol Pharmacol, 1995. 47(2): p. 368-73, which is herein incorporated by reference in its entirety; Sandoval, V., E. L. Riddle, Y. V. Ugarte, G. R. Hanson, and A. E. Fleckenstein, Methamphetamine-induced rapid and reversible changes in dopamine transporter function: an in vitro model. J Neurosci, 2001. 21(4): p. 1413-9, which is herein incorporated by reference in its entirety; Sonders, M. S., S. J. Zhu, N. R. Zahniser, M. P. Kavanaugh, and S. G. Amara, Multiple ionic conductances of the human dopamine transporter: the actions of dopamine and psychostimulants. J Neurosci, 1997. 17(3): p. 960-74, which is herein incorporated by reference in its entirety; Vanderschuren, L. J., A. N. Schoffelmeer, G. Wardeh, and T. J. De Vries, Dissociable effects of the kappa-opioid receptor agonists bremazocine, U69593, and U50488H on locomotor activity and long-term behavioral sensitization induced by amphetamine and cocaine. Psychopharmacology (Berl), 2000. 150(1): p. 35-44, which is herein incorporated by reference in its entirety), morphine appears to differ from oxycodone in its prescription abuse potential (Compton, W. M. and N. D. Volkow, Major increases in opioid analgesic abuse in the United States: Concerns and strategies. Drug Alcohol Depend, 2006. 81(2): p. 103-7, which is herein incorporated by reference in its entirety) and PCP is a non-competitive NMDA antagonist with abuse potential whereas SDZ220-581 is a competitive antagonist with little abuse potential (Baron, S. P. and J. H. Woods, Competitive and uncompetitive N-methyl-D-aspartate antagonist discriminations in pigeons: CGS 19755 and phencyclidine. Psychopharmacology (Berl), 1995. 118(1): p. 42-51, which is herein incorporated by reference in its entirety; Koek, W., J. H. Woods, and F. C. Colpaert, N-methyl-D-aspartate antagonism and phencyclidine-like activity: a drug discrimination analysis. J Pharmacol Exp Ther, 1990. 253(3): p. 1017-25, which is herein incorporated by reference in its entirety). In the third tier, the chosen doses represent a pharmacologically relevant range.

TABLE 3
Agonists

| Drug Class | Drug | Dose | Ref |
|---|---|---|---|
| Psychomotor | Cocaine | 3.0, 5.6, 10.0, 17.0, | 13, 24 |
| stimulants | | 30.0 | |
| | Methamphetamine | 0.3, 1.0 3.0, 5.6 | 27 |
| Opioids | Morphine | 1.0 3.0, 5.6, 10.0 | 14 |
| | Oxycodone | 0.3, 1.0 3.0, 5.6 | 3 |
| Psychotomimetics | Phencyclidine | 1.0 3.0, 5.6, 10.0 | 19, 36 |
| | SDZ 220-581 | 3.0, 5.6, 10.0, 17.0 | 19, 36 |

In this example it is contemplated that a number of potential cocaine therapeutics may be characterized. Since the current invention is a predictive pharmacological model, it contemplates the characterization of the psychoactive profile of a proven therapeutic agent and screening additional compounds for similar psychoactive profile. In our current example, the drugs being characterized are either in clinical trials (modafinil, baclofen; (Vocci, F. and W. Ling, Medications development: successes and challenges. Pharmacol Ther, 2005. 108(1): p. 94-108, which is herein incorporated by reference in its entirety)) or have been shown to be effective in antagonizing the behavioral effects of cocaine (NGB2904, JHW007; (Desai, R. I., T. A. Kopajtic, M. Koffarnus, A. H. Newman, and J. L. Katz, Identification of a dopamine transporter ligand that blocks the stimulant effects of cocaine. J Neurosci, 2005. 25(8): p. 1889-93, which is herein incorporated by reference in its entirety; and Xi, Z. X., A. H. Newman, J. G. Gilbert, A. C. Pak, X. Q. Peng, C. R. Ashby, L. Gitajn, and E. L. Gardner, The Novel Dopamine D(3) Receptor Antagonist NGB 2904 Inhibits Cocaine's Rewarding Effects and Cocaine-Induced Reinstatement of Drug-Seeking Behavior in Rats. Neuropsychopharmacology, 2005, which is herein incorporated by reference in its entirety)). While there are additional compounds available, these compounds provide a range of pharmacological actions which may be contrasted against the effects of cocaine (see Table 4).

TABLE 4
Therapeutics

| Drug Class | Drug | Hypothesized mechanism | Dose | Reference |
|---|---|---|---|---|
| Potential | baclofen | GABA-B agonist | 5.6, 10.0, 17.0, 30.0 | 6, 16, 33, 43 |
| Cocaine | modafinil | Glutaminergic, monaminergic | 5.6, 10.0, 17.0, 30.0 | 9, 40 |
| Therapeutics | JHW007 | Slowly associating DA transporter ligand | 3.0, 5.6, 10.0, 17.0 | 10 |
| | NGB2904 | D3 antagonist | 1.0 3.0, 5.6, 10.0 | 42 |

The mice used in this study will be C57BL/6J males, 60-80 days old. The mice will be shipped from Jackson Laboratories and housed in the animal colony at MPRC for at least two weeks before testing. They will be kept in standard conditions; 12:12 light cycle, 22° C. room temp., water and food ad libitum.

The current invention employs cross-validation. In this case, the Mining Set consists of all groups; drug classes, drugs and doses (n=6 per group). Novel drug class, drug and dose features are identified in this group. The Test Set consists of a second batch of subjects tested across all conditions (n=6 per group) using only the relatively small number of isolated patterns identified in the Mining Set.

In general, mice are injected with one of the drugs detailed in Table 3 and immediately placed in the arena. Drugs and drug doses are assigned so that no two animals from the same cage receive the same drug and dose. The appropriate vehicle control will be used for each drug.

The behavioral data is gathered using the SEE open-field test. The mouse is allowed to freely explore the arena for 45 min while its location is video-tracked and digitized at a rate of 25-30 Hz and spatial resolution of ˜1 cm. The path plot coordinates are smoothed (Hen et al., 2004) and segmented into stops and progression segments (Drai et al., 2000).

In order to determine the effects of the antagonist given alone), the drug is administered 15 min prior to the session followed by a second saline injection given immediately prior to the session. The second injection controls for the two injections needed to assess their antagonist properties against cocaine. In order to determine the effects of the antagonist against cocaine, the antagonist is administered 15 min prior to cocaine. Three doses of each antagonist is tested against the peak stimulant effect of cocaine (doses chosen from antagonist alone study).

The methodology of the current invention for determining a behavioral signature is followed. The composition of the experimental groups is dictated by the level of analysis (i.e., Tier 1, 2 or 3) and whether the drug is given alone or in combination with cocaine.

Step 1, Quantify data points for feature vector: Each data point during the animal's progression is quantified using 7 “features” (variables) (described in Table 1). Step 2, Categorize data points: In each animal, the data points of the path belonging to the progression mode is classified into the cells. Step 3, Analyze differences in relative frequencies: For each cell, the relative frequencies from all animals is tested for experimental group differences using one-way ANOVA. Step 4, Eliminate highly correlated patterns: Using a simple recursive procedure we eliminate correlated patters. Step 5, Validation of identified patterns: All the previous steps are performed in the Mining Set. Finally, the remaining list of cells is tested in the Test Set. The significance is corrected for multiple comparisons using FDR.

Mining Set: The initial set of animals to be tested are given the medium dose of each drug. Since most of these drugs have not been tested in the large open-field arena, adjustments to the dose range may be made following this first round of testing. Following this first round of testing the rest of the doses within each drug are run. The appropriate vehicle controls are run throughout the course of testing in order to account for laboratory effects across time it may take to conduct sessions for the Mining Set.

Test Set: The entire range of doses for each drug tested in the Mining Set may be tested again in the test set. Subjects may be tested in the same manner as the Mining Set. Drugs and drug doses are assigned so that no two animals from the same cage receive the same drug and dose. The appropriate vehicle controls are run throughout the course of testing in order to account for laboratory effects across time.

Drug Class, Drug and Dose Characterizations

Tier One: The one-way ANOVA conducted at Step 3 (above) compares differences across drug class, collapsed across drugs and dose within the class. The individual drugs and doses within each drug class may be pooled in order to discover the behavioral features of a drug class that best characterize drugs within a particular class. The information garnered from this analysis provides a framework to screen test compounds for their general psychoactive properties.

Tier Two: The one-way ANOVA conducted at Step 3 (above) compares differences between drugs within a class, collapsed across dose. The individual doses within each drug may be pooled in order to discover the behavioral features of a particular drug (as compared to other drugs in it's class) that best characterize this particular drug. The information garnered from this analysis provides a framework to screen test compounds for their unique psychoactive properties compared with other drugs in its class.

Tier Three: The one-way ANOVA conducted at Step 3 (above) compares differences between doses within a particular test compound. The information garnered from this analysis provides insight into dose specific behavioral effects. In particular, it determines whether or not low dose effects are quantitatively vs. qualitatively different than high dose effects.

The current invention provides a multi-level and hierarchical methodology that may be applied across various behavioral test models. That is, it tests both very broad properties of the behavior, defined by only a single feature and more refined patterns included in it defined by up to n features (e.g., Table 1 shows 7 features and Table 2 shows 9 features). This may enable the current invention to detect hierarchic differences, such as differences between classes of drugs, between drugs of the same class and between doses of the same drug, all at the same time. The difference between classes may be detected by the more general properties, while the more refined differences may be detected by the more specific patterns.

The magnitude of the data sets utilized by the current invention, show that the current invention may provide significant value in data-mining efforts. Additional analyses that may be performed may include cluster analysis for identifying features most clearly associated with drug class, drug and dose. In addition, once particular behavioral features are identified, template matching may be performed for identifying particular ‘signatures’ that may be valuable in a drug discovery context.

As previously identified, the in silico component of the current invention allows for the mining of the behavioral database for purely investigational purposes and for the characterization of the profile of novel drugs. It is contemplated that the database may be in various forms and the data stored within may be in various formats. The database may include the information related to the drug compounds and therapeutics previously identified and may also include the characterization of other drug classes, including atypical antipsychotics and cannabinoid agonists. It is further contemplated that the database may be mined to determine the effects of characterized therapeutics on various alternative drugs. For example, cocaine therapeutics may be tested for their efficacy against methamphetamine. Further, the current invention may be employed to characterize antagonists (such as, SB277011A and others), cannabinoid antagonists (Arnold, J. C., The role of endocannabinoid transmission in cocaine addiction. Pharmacol Biochem Behav, 2005. 81(2): p. 396-406, which is herein incorporated by reference in its entirety; Beardsley, P. M. and B. F. Thomas, Current evidence supporting a role of cannabinoid CB1 receptor (CB1R) antagonists as potential pharmacotherapies for drug abuse disorders. Behav Pharmacol, 2005. 16(5-6): p. 275-96, which is herein incorporated by reference in its entirety; and Le Foll, B. and S. R. Goldberg, Cannabinoid CB1 receptor antagonists as promising new medications for drug dependence. J Pharmacol Exp Ther, 2005. 312(3): p. 875-83, which is herein incorporated by reference in its entirety), and low dose amphetamine (Negus, S. S. and N. K. Mello, Effects of chronic d-amphetamine treatment on cocaine- and food-maintained responding under a second-order schedule in rhesus monkeys. Drug Alcohol Depend, 2003. 70(1): p. 39-52, which is herein incorporated by reference in its entirety; and Negus, S. S. and N. K. Mello, Effects of chronic d-amphetamine treatment on cocaine- and food-maintained responding under a progressive-ratio schedule in rhesus monkeys. Psychopharmacology (Berl), 2003. 167(3): p. 324-32, which is herein incorporated by reference in its entirety).

The use of the current invention within various behavioral test models may allow the current invention to isolate “intrinsic” properties that depend less on the specific maze. Such properties are likely to be more reliable, as they depend less on the environment. Furthermore, with the increase of the database size the current invention may be able to test any newly isolated pattern in silico in many independent datasets from various drugs and mazes. Such an application of the current invention may allow for the linking of the highly heritable behavioral outcomes to large-scale gene expression database(s) (Kafkafi, N., N. E. Letwin, A. Reiner, D. Yekutieli, Y. Benjamini, N. H. Lee, and G. I. Elmer, Algorithm for discovering multiple heritable movement patterns in mouse behavior and their correlation with gene expression in the brain. Soc for Neurosci Abstracts, 2005. Prog No. 571.16, which is herein incorporated by reference in its entirety; and Letwin, N. E., N. Kafkafi, Y. Benjamini, C. Mayo, B. Frank, N. H. Lee, and G. I. Elmer, Combined application of behavior genetics and microarray analysis to identify regional expression themes and gene-behavior associations. Journal of Neuroscience, 2006. in press, which is herein incorporated by reference in its entirety).

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

### REFERENCES

1. Arnold, J. C., The role of endocannabinoid transmission in cocaine addiction. Pharmacol Biochem Behav, 2005. 81(2): p. 396-406.
2. Baron, S. P. and J. H. Woods, Competitive and uncompetitive N-methyl-D-aspartate antagonist discriminations in pigeons: CGS 19755 and phencyclidine. Psychopharmacology (Berl), 1995. 118(1): p. 42-51.
3. Beardsley, P. M., M. D. Aceto, C. D. Cook, E. R. Bowman, J. L. Newman, and L. S. Harris, Discriminative stimulus, reinforcing, physical dependence, and antinociceptive effects of oxycodone in mice, rats, and rhesus monkeys. Exp Clin Psychopharmacol, 2004. 12(3): p. 163-72.
4. Beardsley, P. M. and B. F. Thomas, Current evidence supporting a role of cannabinoid CB1 receptor (CB1R) antagonists as potential pharmacotherapies for drug abuse disorders. Behav Pharmacol, 2005. 16(5-6): p. 275-96.
5. Benjamini, Y., Drai, D., Elmer, G., Kafkafi, N. & Golani, I. (2001) Controlling the false discovery rate in behavior genetics research. Behav Brain Res 125(1-2), 279-284.
6. Benjamini Y. & Yekutieli D. (2005) False discovery rate-adjusted multiple confidence intervals for selected parameters. J Amer Stat Assoc 100, 71.
7. Brebner, K., A. R. Childress, and D. C. Roberts, A potential role for GABA(B) agonists in the treatment of psychostimulant addiction. Alcohol Alcohol, 2002. 37(5): p. 478-84.
8. Broadhurst, P. L. (1975). The Maudsley reactive and nonreactive strains of rats: A survey. Behav Genet 5, 299-319.
9. Bruijn, L. I., Becher, M. W., Lee, M. K., Anderson, K. L., Jenkins, N. A., Copeland, N. G., Sisodia, S. S., Rothstein, J. D., Borchelt, D. R., Price, D. L. & Cleveland, D. W. (1997) ALS-linked SOD1 mutant G85R mediates damage to astrocytes and promotes rapidly progressive disease with SOD1-containing inclusions. Neuron 18, 327-338.
10. Bruijn, L. I., Miller, T. M. & Cleveland, D. W. (2004) Unraveling the mechanisms involved in motor neuron degeneration in ALS. Annu Rev Neurosci 27, 723-749.
11. Chesler, E. J., Wilson, S. G., Lariviere, W. R., Rodriguez-Zas, S. L. & Mogil, J. S. (2002) Influences of laboratory environment on behavior. Nature Neurosci 5, 1101-1102.
12. Chiu, A. Y., Zhai, P., Dal Canto, M. C., Peters, T. M., Kwon, Y. W., Prattis, S. M. & Gurney, M. E., (1995) Age-dependent penetrance of disease in a transgenic mouse model of familial amyotrophic lateral sclerosis. Mol Cell Neurosci 6, 349-362.
13. Collins, G. T., J. M. Witkin, A. H. Newman, K. A. Svensson, P. Grundt, J. Cao, and J. H. Woods, Dopamine agonist-induced yawning in rats: a dopamine D3 receptor-mediated behavior. J Pharmacol Exp Ther, 2005. 314(1): p. 310-9.
14. Compton, W. M. and N. D. Volkow, Major increases in opioid analgesic abuse in the United States: Concerns and strategies. Drug Alcohol Depend, 2006. 81(2): p. 103-7.
15. Crabbe, J. C., Wahlsten, D. & Dudek, B. C. (1999) Genetics of mouse behavior: Interactions with laboratory environment. Science 284 (5420), 1670-1672.
16. Dackis, C. A., K. M. Kampman, K. G. Lynch, H. M. Pettinati, and C. P. O'Brien, A double-blind, placebo-controlled trial of modafinil for cocaine dependence. Neuropsychopharmacology, 2005. 30(1): p. 205-11.
17. Desai, R. I., T. A. Kopajtic, M. Koffarnus, A. H. Newman, and J. L. Katz, Identification of a dopamine transporter ligand that blocks the stimulant effects of cocaine. J Neurosci, 2005. 25(8): p. 1889-93.
18. Drai, D., Benjamini, Y. & Golani, I. (2000) Statistical discrimination of natural modes of motion in rat exploratory behavior. J Neurosci Methods 96(2), 119-31.
19. Drai, D., Kafkafi, N., Benjamini, Y., Elmer, G. I. & Golani, I. (2001) Rats and mice share common ethologically relevant parameters of exploratory behavior. Behav Brain Res 125(1-2), 133-140.
20. Drai, D. & Golani, I. (2001) SEE: a tool for the visualization and analysis of rodent exploratory behavior. Neurosci Biobehav Rev, 25 (5), 409-426.
21. Derave, W., Van Den Bosch, L., Lemmens, G., Eijnde, B. O., Robberecht, W. & Hespel, P. (2003) Skeletal muscle properties in a transgenic mouse model for amyotrophic lateral sclerosis: effects of creatine treatment. Neurobiol Dis 13 (3), 264-72.
22. Elmer, G. I., D. A. Gorelick, S. R. Goldberg, and R. B. Rothman, Acute sensitivity vs. context-specific sensitization to cocaine as a function of genotype. Pharmacol Biochem Behav, 1996. 53(3): p. 623-8.
23. Elmer, G. I., J. O. Pieper, S. S. Negus, and J. H. Woods, Genetic variance in nociception and its relationship to the potency of morphine-induced analgesia in thermal and chemical tests. Pain, 1998. 75(1): p. 129-40.
24. Elmer, G. I., J. O. Pieper, J. Levy, M. Rubinstein, M. J. Low, D. K. Grandy, and R. A. Wise, Brain stimulation and morphine reward deficits in dopamine D2 receptor-deficient mice. Psychopharmacology (Berl), 2005. 182(1): p. 33-44.
25. Fischer, L. R., Culver, D. G., Tennant, P., Davis, A. A., Wang, M., Castellano-Sanchez, A., Khan, J., Polak, M. A. & Glass, J. D. (2004) Amyotrophic lateral sclerosis is a distal axonopathy: evidence in mice and man. Exp Neurol 185, 232-240.
26. Grammer, M., Kuchay, S. Chishti, A. & Baudry, M. (2005) Lack of phenotype for LTP and fear conditioning learning in calpain 1 knock-out mice. Neurobiol Learn Mem 84(3), 222-227.
27. Gurney, M. E., Pu, H., Chiu, A. Y., Dal Canto, M. C., Polchow, C. Y., Alexander, D. D., Caliendo, J., Hentati, A., Kwon, Y. W., Deng, H. X. et al. (1994) Motor neuron degeneration in mice that express a human Cu,Zn superoxide dismutase mutation. Science 264(5166), 1772-1775.
28. Haney, M., C. L. Hart, and R. W. Foltin, Effects of Baclofen on Cocaine Self-Administration: Opioid- and Nonopioid-Dependent Volunteers. Neuropsychopharmacology, 2006.
29. Heidbreder, C. A. and J. J. Hagan, Novel pharmacotherapeutic approaches for the treatment of drug addiction and craving. Curr Opin Pharmacol, 2005. 5(1): p. 107-18.
30. Hen, I., Sakov, A., Kafkafi, N., Golani, I. & Benjamini, Y. (2004) The dynamics of spatial behavior: how can robust smoothing techniques help? J Neurosci Methods 133(1-2), 161-172.
31. Horev, G., Benjamini, Y., Sakov, A. & Golani, I. (in press) Estimating wall guidance and attraction in mouse free locomotor behavior. Genes Brain Behav.
32. Howland, D. S., Liu, J., She, Y., Goad, B., Maragakis, N. J., Kim, B., Erickson, J., Kulik, J., DeVito, L., Psaltis, G., DeGennaro, L. J., Cleveland, D. W. & Roth-stein, J. D., (2002). Focal loss of the glutamate transporter EAAT2 in a transgenic rat model of SOD1 mutant-mediated amyotrophic lateral sclerosis (ALS). Proc Natl Acad Sci USA 99, 1604-1609.
33. Jayaram, N., C. Mayo, H. Guard, and G. I. Elmer, Discriminating the effects of typical from atypical antipsychotics in the C57BL/6J mouse. Soc for Neurosci Abstracts, 2003. Prog No. 385.21.
34. Kafkafi N, Mayo C, Drai D, Golani I, Elmer G. (2001) Natural segmentation of the locomotor behavior of drug-induced rats in a photobeam cage. J Neurosci Methods 109(2), 111-21.
35. Kafkafi, N. (2003) Extending SEE for large-scale phenotyping of mouse open-field behavior. Behav Res Methods Instrum Comput 35(2), 294-301.
36. Kafkafi, N., Lipkind, D., Benjamini. Y., Mayo, C. L., Elmer, G. I. & Golani I. (2003a) SEE locomotor behavior test discriminates C57BL/6J and DBA/2J mouse inbred strains across laboratories and protocol conditions. Behav Neurosci 117(3), 464-477.
37. Kafkafi, N., Pagis, M., Lipkind, D., Mayo, C. L., Benjamini, Y., Elmer, G. I. & Golani, D. (2003b). Darting behavior: a quantitative movement pattern for discrimination and replicability in mouse locomotor behavior. Behav Brain Res 142(1-2), 193-205.
38. Kafkafi, N. & Elmer G. I. (2005) Texture of locomotor path: a replicable characterization of a complex behavioral phenotype. Genes Brain Behav 4(7), 431-443.
39. Kafkafi, N., Benjamini, Y., Sakov, A., Elmer, G. I. & Golani, I. (2005) Genotype-environment interactions in mouse behavior: a way out of the problem. Proc Natl Acad Sci USA 102(12), 4619-4624.
40. Kafkafi, N. and G. I. Elmer, Activity density in the open field: a measure for differentiating the effect of psychostimulants. Pharmacol Biochem Behav, 2005. 80(2): p. 239-49.
41. Kafkafi, N., N. E. Letwin, A. Reiner, D. Yekutieli, Y. Benjamini, N. H. Lee, and G. I. Elmer, Algorithm for discovering multiple heritable movement patterns in mouse behavior and their correlation with gene expression in the brain. Soc for Neurosci Abstracts, 2005. Prog No. 571.16.
42. Koek, W., J. H. Woods, and F. C. Colpaert, N-methyl-D-aspartate antagonism and phencyclidine-like activity: a drug discrimination analysis. J Pharmacol Exp Ther, 1990. 253(3): p. 1017-25.
43. Kuribara, H. and S. Tadokoro, Reverse tolerance to ambulation-increasing effects of methamphetamine and morphine in 6 mouse strains. Jpn J Pharmacol, 1989. 49(2): p. 197-203.
44. Le Foll, B. and S. R. Goldberg, Cannabinoid CB1 receptor antagonists as promising new medications for drug dependence. J Pharmacol Exp Ther, 2005.312(3): p. 875-83.
45. Leppanen, P. K., Ewalds-Kvist, S. B. & Selander, R. K. (2005) Mice selectively bred for open-field thigmotaxis: life span and stability of the selection trait. J Gen Psychol 132(2), 187-204, (hereinafter “Leppanen et al., 2005”).
46. Letwin, N. E., N. Kafkafi, Y. Benjamini, C. Mayo, B. Frank, N. H. Lee, and G. I. Elmer, Combined application of behavior genetics and microarray analysis to identify regional expression themes and gene-behavior associations. Journal of Neuroscience, 2006. in press.
47. Lipkind, D., Sakov, A., Kafkafi, N., Elmer, G. I., Benjamini, Y. & Golani, I. (2004) New replicable anxiety-related measures of wall vs center behavior of mice in the open field. J Appl Physiol 97(1), 347-359.
48. Matsumoto, A., Okada, Y., Nakamichi, M., Nakamura, M., Toyama, Y., Sobue, G., Nagai, M., Aoki, M., Itoyama, Y. & Okano, H. (2006) Disease progression of human SOD1 (G93A) transgenic ALS model rats. J Neurosci Res 83 (1), 119-33.
49. Negus, S. S. and N. K. Mello, Effects of chronic d-amphetamine treatment on cocaine- and food-maintained responding under a second-order schedule in rhesus monkeys. Drug Alcohol Depend, 2003. 70(1): p. 39-52.
50. Negus, S. S. and N. K. Mello, Effects of chronic d-amphetamine treatment on cocaine- and food-maintained responding under a progressive-ratio schedule in rhesus monkeys. Psychopharmacology (Berl), 2003. 167(3): p. 324-32.
51. Perez, F. A. & Palmiter, R. D. (2005) Parkin-deficient mice are not a robust model of parkinsonism. Proc Natl Acad Sci USA 102(6), 2174-2179.
52. Pifl, C., H. Drobny, H. Reither, O. Hornykiewicz, and E. A. Singer, Mechanism of the dopamine-releasing actions of amphetamine and cocaine: plasmalemmal dopamine transporter versus vesicular monoamine transporter. Mol Pharmacol, 1995. 47(2): p. 368-73.
53. Ramos, A., Berton, O., Mormede, P. & Chaouloff, F. (1997) A multiple-test study of anxiety-related behaviours in six inbred rat strains. Behav Brain Res 85(1), 57-69.
54. Roberts, D. C., Preclinical evidence for GABAB agonists as a pharmacotherapy for cocaine addiction. Physiol Behav, 2005. 86(1-2): p. 18-20.
55. Rothstein, J. D., Patel, S., Regan, M. R., Haenggeli, C., Huang, Y. H., Bergles, D. E., Jin, L, Dykes Hoberg, M., Vidensky, S., Chung, D. S., Toan, S. V., Bruijn, L, I, Su, Z. Z., Gupta, P. & Fisher P B. (2005) Beta-lactam antibiotics offer neuroprotection by increasing glutamate transporter expression. Nature 433, 73-77.
56. Sandoval, V., E. L. Riddle, Y. V. Ugarte, G. R. Hanson, and A. E. Fleckenstein, Methamphetamine-induced rapid and reversible changes in dopamine transporter function: an in vitro model. J Neurosci, 2001. 21(4): p. 1413-9.
57. Shaw, C. A. & Wilson, J. M. B. (2006) Environmental toxicity and ALS: novel iinsights from an animal model of ALS-PDC, in Amyotrophic Lateral Sclerosis. Mitsumoto, H., Przedborski, S. and Gordon, P. H., (Eds), New York: Taylor & Francis, 435-448.
58. Shoptaw, S., X. Yang, E. J. Rotheram-Fuller, Y. C. Hsieh, P. C. Kintaudi, V. C. Charuvastra, and W. Ling, Randomized placebo-controlled trial of baclofen for cocaine dependence: preliminary effects for individuals with chronic patterns of cocaine use. J Clin Psychiatry, 2003. 64(12): p. 1440-8.
59. Sonders, M. S., S. J. Zhu, N. R. Zahniser, M. P. Kavanaugh, and S. G. Amara, Multiple ionic conductances of the human dopamine transporter: the actions of dopamine and psychostimulants. J Neurosci, 1997. 17(3): p. 960-74.
60. Tapocik, J., N. Jayaram, H. Guard, C. Mayo, C. Stamps, X.-M. Gao, C. Tamminga, and G. I. Elmer, Antipsychotic effects against competitive and non-competitive NMDA antagonists in the mouse. Soc for Neurosci Abstracts, 2005. Prog No. 676.11.
61. Vanderschuren, L. J., A. N. Schoffelmeer, G. Wardeh, and T. J. De Vries, Dissociable effects of the kappa-opioid receptor agonists bremazocine, U69593, and U50488H on locomotor activity and long-term behavioral sensitization induced by amphetamine and cocaine. Psychopharmacology (Berl), 2000.150(1): p. 35-44.
62. Vocci, F. and W. Ling, Medications development: successes and challenges. Pharmacol Ther, 2005. 108(1): p. 94-108.
63. Wahlsten, D., Rustay, N. R., Metten, P. & Crabbe, J. C. (2003) In search of a better mouse test. Trends Neurosci 26(3), 132-136.
64. Weydt, P., Hong, S. Y., Kliot, M. and Moller, T. (2003) Assessing disease onset and progression in the SOD1 mouse model of ALS. Neuroreport 14 (7), 1051-4.
65. Willner P (1991) Methods for assessing the validity of animal models of human psychopathology. In: Animal models in psychiatry, I, 18 Edition (Boulton A A, Baker G B, Martin-Iversen M T, eds), pp 1-24. New Jersey: Humana Press.
66. Wisor, J. P., S. Nishino, I. Sora, G. H. Uhl, E. Mignot, and D. M. Edgar, Dopaminergic role in stimulant-induced wakefulness. J Neurosci, 2001. 21(5): p. 1787-94.
67. Xi, Z. X., J. G. Gilbert, A. C. Pak, C. R. Ashby, Jr., C. A. Heidbreder, and E. L. Gardner, Selective dopamine D3 receptor antagonism by SB-277011A attenuates cocaine reinforcement as assessed by progressive-ratio and variable-cost-variable-payoff fixed-ratio cocaine self-administration in rats. Eur J Neurosci, 2005. 21(12): p. 3427-38.
68. Xi, Z. X., A. H. Newman, J. G. Gilbert, A. C. Pak, X. Q. Peng, C. R. Ashby, L. Gitajn, and E. L. Gardner, The Novel Dopamine D(3) Receptor Antagonist NGB 2904 Inhibits Cocaine's Rewarding Effects and Cocaine-Induced Reinstatement of Drug-Seeking Behavior in Rats. Neuropsychopharmacology, 2005.

What is claimed is:

1. A method for identifying behavioral signatures for drug discovery, comprising:
identifying a set of data points corresponding to a physical location of an exploratory path of a test subject;
defining a plurality of behavioral patterns corresponding to a plurality of features and an interval for each of the features;
associating each data point with one of the plurality of behavioral patterns, thereby creating an endpoint;
determining the relative frequency for each of the plurality of behavioral patterns and identifying a set of endpoints; and
identifying a behavioral signature defined by the set of endpoints.

2. The method recited in claim 1, wherein the step of identifying data points further includes the steps of monitoring the exploratory path and storing the physical locations corresponding to the exploratory path.

3. The method recited in claim 1, wherein the exploratory path may be from a behavioral test selected from the group consisting of an open-field maze, photobeam box, plus maze and water maze.

4. The method recited in claim 1, wherein each of the plurality of features includes a range defined by one or more intervals.

5. The method recited in claim 4, wherein each of the plurality of features is defined over at least one of a short time period and a long time period.

6. The method recited in claim 1, wherein each of the plurality of behavioral patterns corresponds with a cell and a plurality of cells define a feature space.

7. The method recited in claim 1, further comprising reducing the number of behavioral patterns prior to identifying the behavioral signature.

8. The method recited in claim 7, further comprising reducing the number of behavioral patterns based on the relative frequency determined for the behavioral pattern.

9. The method recited in claim 8, further comprising retaining only those behavioral patterns in which a difference between the experimental groups is higher than a predetermined value.

10. The method recited in claim 7, further comprising:
retaining a behavioral pattern having a highest significance;
discarding any behavioral pattern highly correlated with it; and
repeating the retaining and discarding steps for any remaining behavioral patterns.

11. The method recited in claim 1, further comprising validating the identified behavioral signature by testing it with respect to a test set.

12. The method recited in claim 1, applied in a behavior genetics application for identifying heritable traits.

13. The method recited in claim 1, applied in a pharmacological application for identifying behavioral effects of a drug.

14. The method recited in claim 1, applied in at least one of a disease application for diagnosing the presence of disease in a test subject and a therapeutic application for determining the effectiveness of a therapy.

15. A system for identifying behavioral signatures for drug discovery, comprising:
a video camera for monitoring physical locations of an exploratory path of a test subject allowed to explore a pen for a period of time;
a computer communicatively coupled with the video camera, the computer including a storage device for storing the monitored locations, wherein the computer determines a relative frequency for a plurality of behavioral patterns, each behavioral pattern corresponding to a unique combination of defined intervals of a plurality of features and associates each of the behavioral patterns with an endpoint to form a set of endpoints that determine a behavioral signature.

16. The system recited in claim 15, wherein the computer further reduces the number of behavioral patterns prior to identifying the behavioral signature based on the determined relative frequency for each behavioral pattern.

17. The system recited in claim 16, wherein the computer retains only those behavioral patterns in which a difference between experimental groups is higher than a predetermined value.

18. The system recited in claim 16, wherein the computer further:
retains a behavioral pattern having a highest significance;
discards any behavioral pattern highly correlated with it; and
repeats the retaining and discarding of behavioral patterns for any remaining behavioral patters.

19. The system recited in claim 15, wherein the computer is used to validate the identified behavioral signature by testing it with respect to a test set.

20. The system recited in claim 15, used in a behavior genetics application for identifying heritable traits.

21. The system recited in claim 9 used in a pharmacological application for identifying a drug's behavioral effects.

22. A method for diagnosing a disease in a test subject, comprising:
determining the behavioral signature of the test subject; and
comparing the behavioral signature of the test subject against the behavioral signature of the disease.

23. The method of claim 22, further comprising the step of determining the behavioral signature of the disease prior to making the comparison.

24. A method for testing a therapy for a disease, comprising:
determining a behavioral signature of the disease; and
evaluating the therapy for its therapeutic effectiveness in addressing the behavioral signature of the disease.

25. The method of claim 24, further comprising the step of determining the behavioral signature of the therapy.

26. The method of claim 24, further comprising the step of evaluating the behavioral signature of the therapy for its therapeutic effectiveness in addressing the behavioral signature of the disease.

27. A method of drug discovery, comprising:
obtaining a first drug and determining a first behavioral signature of the first drug; and
comparing the first behavioral signature of the first drug with a second behavioral signature of a second drug, wherein the information regarding the second drug and the second behavioral signature is stored within a repository,
classifying the first drug based upon a significant correlation between the first behavioral signature and the second behavioral signature.

28. The method of claim 27, further comprising the step of building a repository of a plurality of drugs and their corresponding behavioral signature.

29. The method of claim 28, further comprising the step of adding the first drug to the repository when the first behavioral signature is known.

30. The method of claim 27, further comprising:
determining a behavioral signature of a therapeutic; and
comparing the behavioral signature of the therapeutic with the second behavioral signature.
